(19) United States
(12) Patent Application Publication
(10) Pub. No.: US 2026/0151191 A1
(43) Pub. Date: Jun. 4, 2026

(54) FIBER SENSORS FOR INTERVENTIONAL TOOLS

(71) Applicant: DEEPSIGHT TECHNOLOGY, INC., Santa Clara, CA (US)

(72) Inventors: Jennifer YOUNG, Green Bay, WI (US); Linhua XU, University City, MO (US); Jiangang ZHU, St. Louis, MO (US)

(21) Appl. No.: 19/403,738

(22) Filed: Nov. 28, 2025

Related U.S. Application Data
(60) Provisional application No. 63/726,444, filed on Nov. 29, 2024.

(51) Int. Cl. A61B 34/20

(52) U.S. Cl. CPC A61B 34/20; A61B 2034/2063; A61B 2034/2065

(57) ABSTRACT

Systems described herein may be provided to facilitate and improve medical guidance. Such systems may include one or more instruments or medical devices, each equipped with one or more optical transducers. Medical guidance systems may further include ultrasound probes. The optical transducers may be configured to respond to ultrasound signals emitted by the ultrasound probes. The medical guidance systems are configured to determine location information according to the response of the optical transducers.

### RELATED APPLICATIONS

This application claims priority to U.S. Provisional Application No. 63/726,444, filed on Nov. 29, 2024, the entire contents of which are hereby incorporated by reference.

This disclosure refers to U.S. Pat. No. 12,025,489, titled “FIBER-OPTICAL SENSOR SYSTEM FOR ULTRASOUND SENSING AND IMAGING,” issued on Jul. 2, 2024, to patent Ser. No. 18/382,984, titled TRANSPONDER TRACKING AND ULTRASOUND IMAGE ENHANCEMENT and filed on Oct. 23, 2023, to patent application Ser. No. 18/609,378 , titled “FIBER-OPTICAL SENSOR ARRAY FOR SENSING AND IMAGING,” filed on Mar. 19, 2024, to patent application Ser. No. 18/698,193 , titled “ULTRASOUND BEACON VISUALIZATION WITH OPTICAL SENSORS,” and filed on Oct. 7, 2022, to patent application Ser. No. 18/685,985 , titled “MULTI-DIMENSIONAL SIGNAL DETECTION WITH OPTICAL SENSORS,” and filed on Feb. 23, 2024, to patent application Ser. No. 18/597,493, filed on Mar. 6, 2024, titled “Mixed Array Imaging Probe,” to U.S. application Ser. No. 17/990,596, filed on Nov. 18, 2022 and titled “Mixed Ultrasound Transducer Arrays,” and U.S. application Ser. No. 17/244,605, filed on Apr. 29, 2021 titled “Modularized Acoustic Probe, each of which is incorporated herein by reference in its entirety.

### TECHNICAL FIELD

This disclosure relates generally to the field of visualizing and/or tracking objects and visualizing the area around the object using an ultrasound beacon or transponder signal. More particularly, this disclosure relates to the use of ultrasound based tool sensor for tracking of objects and visualizing from objects in various surgical and interventional procedures.

### BACKGROUND

Acoustic imaging is used in various industries including medical imaging. For example, acoustic imaging technology may be used to visualize objects (e.g., needles, catheters, guidewires) used in clinical procedures such as biopsy, drug delivery, catheterization, device implantation, etc. Using acoustic imaging for medical applications offers several advantages. For instance, acoustic imaging such as ultrasound imaging is a non-invasive form of imaging. Additionally, ultrasound imaging uses ultrasound signals which are known to have remarkable penetration depth.

Some existing acoustic imaging technology uses piezoelectric (PZT) transducers to visualize and track objects (e.g., needles, catheters, drug delivery pumps, etc.) and/or images from the perspective of the device. However, PZT transducers generally have low output. Imaging technology including PZT transducers often require bulky circuits. Therefore, it may be challenging to use PZT transducers for medical applications because of the size limitations (e.g., physical size). Other medical tool tracking techniques may include needle guides secured to an ultrasound probe or transducer, magnetic, electromagnetic or GPS system. These types of systems may need additional equipment and/or external energy fields, which may make it challenging to incorporate these technologies into medical procedures. Further, needle guides limit the angle of approach for a clinician and thus may not be adaptable to a range of procedures.

Accordingly, there is a need for new and improved compact technology with high sensitivity to visualize and track objects, provide for enhanced imaging at the location of a tool within an insonified region during a procedure, and/or providing additional imaging from the perspective of the tool, especially for interventional medical procedures.

### SUMMARY

Systems and methods for visualizing position of an object are described herein. In some variations, a method for visualizing position of an object may include emitting acoustic beamforming pulses and acoustic beacon pulses from an ultrasound array, receiving acoustic beamforming signals corresponding to the acoustic beamforming pulses and acoustic beacon signals corresponding to the acoustic beacon pulses with one or more optical sensors arranged on the object, generating an ultrasound image based on the acoustic beamforming signals, and generating an object indicator based on the acoustic beacon signals.

In some aspects, the techniques described herein relate to a system for medical device tracking, including: an ultrasound transducer configured to generate ultrasound beacon signals; a first instrument including a first optical ultrasound transducer configured to receive the ultrasound beacon signals and generate first optical signals corresponding to the ultrasound beacon signals; a second instrument including a second optical ultrasound transducer configured to receive the ultrasound beacon signals and generate second optical signals corresponding to the ultrasound beacon signals; at least one processor configured to: obtain first location signals corresponding to the first optical signals; obtain second location signals corresponding to the second optical signals; cause display of a first location indicator corresponding to a first location of the first instrument and a second location indicator corresponding to a second location of the second instrument.

In some aspects, the techniques described herein relate to a system for medical device guidance, including: an ultrasound transducer configured to generate ultrasound beacon signals; an instrument including an optical ultrasound transducer configured to receive the ultrasound beacon signals and generate optical signals corresponding to the ultrasound beacon signals; at least one processor configured to: obtain location signals corresponding to the optical signals; cause display of a location indicator corresponding to a location of the instrument.

In some aspects, the techniques described herein relate to a system for ablation tool guidance, including: an ultrasound transducer configured to generate ultrasound beacon signals; an instrument including an optical ultrasound transducer configured to receive the ultrasound beacon signals and generate optical signals corresponding to the ultrasound beacon signals; at least one processor configured to: obtain location signals corresponding to the optical signals; cause display of a location indicator corresponding to a location of the instrument.

### BRIEF DESCRIPTION OF THE DRAWINGS

FIG. 1A is an exemplary variation of a system for ultrasound beacon visualization of an object.

FIG. 1B illustrates a processing system consistent with embodiments hereof.

FIG. 2(a)-2(c) illustrate examples of an optical sensor detecting acoustic signals as a point sensor or a line sensor.

FIG. 3A illustrates a portion of an exemplary variation of a system with a needle and an optical sensor attached to the needle to track the needle and/or determine a position of the needle.

FIG. 3B illustrates a portion of an exemplary variation of a system with a needle and two optical sensors attached to the needle to track the needle and/or determine a position of the needle.

FIG. 4 is an exemplary schematic illustrating positions of elements configured to emit acoustic beacon pulses and illustrating position of an optical sensor in a Cartesian coordinate system so as to locate an object.

FIGS. 5 and 6 show example methods for transponder tracking and ultrasound image enhancement.

FIG. 7 is a flow chart of an embodiment of a method for generating an ultrasound image from the transponder sensed signals

FIG. 8 is an exemplary schematic of an instrument sensor within the imaging plane and an instrument sensor outside of the imaging plane

FIGS. 9 and 10 illustrate the use of embodiments, methods, devices and systems described herein to improve a transjugular intrahepatic portosystemic shunt (TIPS) procedure

FIG. 11A and FIG. 11B illustrate examples of on-tool optical based location tracking and imaging

FIG. 12 illustrates an axial view of a patient abdomen.

FIGS. 13A and 13B show examples of tracking an instrument with an on-tool optical based ultrasound transducer/sensor.

FIG. 14A illustrates a moving shot ablation technique.

FIG. 14B shows the thyroid amid adjacent structures that can be damaged during an ablation procedure.

FIGS. 15A-15C are ultrasound images of a kidney.

FIG. 16 illustrates instruments that may be used in a nephrostomy procedure.

FIG. 17 illustrates an instrument that may be employed with rendezvous procedures.

FIG. 18 illustrates a snare that has been guided to the interior of a patient kidney.

FIG. 19 illustrates a needle under ultrasound being guided to the interior of the kidney to rendezvous with the snare.

FIG. 20 is a schematic of the kidney illustrating the rendezvous of the needle tool and the snare tool.

FIG. 21 illustrates a guide wire that has been inserted into the kidney via the needle tool and capture by the snare tool.

FIG. 22A illustrates a patent vein.

FIG. 22B illustrates an occluded vein.

FIG. 23 illustrates the use of a pair of blunt dissection surgical tools, each equipped with an on-tool optical based ultrasound transducer.

### DETAILED DESCRIPTION

Non-limiting examples of various aspects and variations of the invention are described herein and illustrated in the accompanying drawings.

Systems, devices, and methods for ultrasound beacon or transponder visualization with on-tool ultrasound based optical sensors are described herein. For example, the technology described herein may track, locate, monitor and provide guidance for objects, such as surgical instruments, during medical procedures using acoustic signals with optical sensors. The technology described herein is compact in size and has high sensitivity, thereby making it viable for various medical applications such as medical imaging for tracking objects (e.g., needle, catheter, introducers, guidewire, etc.) during biopsy, drug delivery, catheterization, etc. Further, the technology described herein may be used to enhance ultrasound images captured by an external transducer using the information captured by the on-tool ultrasound based optical sensors as well as to provide imaging from the perspective of the on-tool sensors.

Object visualization in medical applications may be an important aspect for performing various medical procedures in a safe and reliable manner. For instance, medical practitioners visualize and track a needle tip while administering anesthesia or therapeutics, performing biopsies and other procedures, to ensure safety. In such instances, adequate needle tip visualization may help prevent unintentional vascular, neural, or visceral injury. Similarly, it may be helpful to visualize needles or introducers and/or other related tools when performing medical procedures such as the Seldinger technique or catheterization to gain access to blood vessels and/or other organs in a safe manner.

Some existing technologies use ultrasound imaging for guidance during medical procedures, to visualize anatomical structures of interest as well as advancing needles or other tools. However, there are several drawbacks associated with existing ultrasound imaging technology for medical applications. Traditional technology uses imaging probes that emit ultrasound waves. But, because of the smooth surface of the needle or other tool or it's angle with respect to the transmitted ultrasound waves, the incident ultrasound waves reflected from the needle surface may be steered away from the receiving direction or not have a large enough reflecting surface. This may make the reflected waves too weak to be detected easily. Altering the needle or needle tip to be more echogenic still presents limitations depending upon the angle with respect to the emitted ultrasound signals, the anatomy in the area of the procedure and/or the artifacts created by the anatomy of the area being imaged. Some conventional needle tracking techniques include the use of needle guides secured to ultrasound probes, magnetic, electromagnetic, or GPS systems, and others. Several of these techniques may need, additional equipment, or external energy fields making it challenging to incorporate the technology during medical procedures. For other procedures, due to the complexity, ionizing imaging devices must be used such as CT or fluoroscopy.

In contrast, the technology described herein using on-tool ultrasound based optical sensors is compact in size and has high sensitivity. In some variations, a method for visualizing a position of an object (e.g., needles, introducers, snares, guidewires, wires, catheters, endoscopes, blunt dissection tools, stylets, introducers, trocars, and any other surgical, diagnostic, or medical instrument) may use an ultrasound array including one or more optical sensors arranged on the object (e.g., coupled to the object, integrally formed with the object, etc.). The method may include emitting acoustic beamforming pulses and acoustic beacon pulses from an ultrasound array, e.g., such as an external ultrasound probe.

As used herein, acoustic beacon (or transponder) signals refer to signals received by on-tool ultrasound based optical sensors that are integrated with a surgical or non-interventional tool for location, tracking, guidance, and tool visualization. Acoustic beacon signals may be transmitted by an external ultrasound probe and/or by any other suitable ultrasound probe. Acoustic beamforming signals refers to signals used for the purpose of image generation. Acoustic beamforming signals may be received by either on-tool ultrasound based optical sensors or by other ultrasound probes (e.g., external probes). In some embodiments, acoustic beacon and acoustic beamforming signals may be different types of signals. In some embodiments, acoustic beamforming signals may be used as acoustic beacon signals and acoustic beacon signals may be use as acoustic beamforming signals.

Acoustic beacon signals that correspond to the acoustic beacon pulses generated by an ultrasound transducer may be received with one or more optical sensors integrated with a medical instrument, for example, a surgical tool or a non-interventional tool. Additionally or alternatively, an object or location indicator (e.g., graph, trace, grid, and/or other visual indicators) may be generated based on the received acoustic beacon signals. The object or location indicator may be representative of the current position of the object, e.g., the medical instrument. Furthermore, a current position of the object may be stored/or tracked over time to enable display and/or other visualization that communicates a trajectory of the object's position. In addition, the location indicator may include an in-plane indicator representing whether or not the tracked object is within the ultrasound imaging plane.

An ultrasound image (or images) may be generated based on the acoustic beamforming signals. In embodiments, such images may be generated from acoustic beamforming signals received by, for example, an external ultrasound transducer. These images may be enhanced or improved by information captured from acoustic beamforming signals received by on-tool ultrasound based optical sensors. Finally, in further embodiments, acoustic beamforming signals may be used to create an image from the perspective of on-tool ultrasound based optical sensors located within the body (also referred to herein as tool-perspective imagery), thus providing further context of the anatomy at the location of the procedure.

By “perspective” it is meant that ultrasound images may be generated using the one or more on-tool ultrasound based optical sensors as a point of origin. In a traditional ultrasound image, provided from the perspective of an external ultrasound transducer probe, the probe head is the point of origin and the image represents objects (e.g., tissue) arranged in the acoustic field of view of the probe. Images “from the perspective” of an optical sensor may be images within the acoustic field of view of the optical sensor and have, for example, an origin point within the body of a patient.

Fiber optical sensors or transducers consistent with the present disclosure are able to provide ultrasound receivers with high sensitivity, broad bandwidth, and a wide acceptance angle and do not require the electrical components needed for electro-mechanical transducers. With these characteristics, fiber optical sensors are able to sense harmonic or scattered signals that existing technologies cannot sense. Further, fiber optical sensors consistent with the present disclosure may be compact, low cost, and may contribute to a scalable sensor system. Embodiments hereof include fiber optical sensors configured to detect acoustic signals. Such fiber optical sensors may be disposed at the end of an optical fiber, adjacent an end of an optical fiber or at a diagnostic or therapeutic relevant location on the medical device to create a sensor fiber. Fiber optical sensors include resonant structures, including, but not limited to Fabry-Perot (FP) resonators, optical cavity resonators, whispering-gallery-mode resonators, and photonic crystal resonators; optical interferometers, including but not limited to MZI, phase-shift coherent interferometers, self-mixing interferometers; acoustically responsive fiber end facets; and acoustic induced birefringent polarization sensors. Fiber optical sensors disclosed herein may further include fiber Bragg gratings (FBG) as part of the acoustically responsive structures.

Acoustically responsive fiber end facets may comprise a substrate suitable for adding various microstructures to enhance the response of the fiber sensor to acoustic signals. Such microstructures may be acoustically responsive structures such as metasurfaces including patterns of small elements (e.g., having a size less than approximately one wavelength of the optical signal) arranged to change the wavefront shape of the acoustic signals and maximize the detection of acoustic signals, acoustically responsive low-dimensional materials with special optomechanical features that are more prone to deformation, and plasmonic structures patterned to amplify light-matter interactions. In addition to operating as an optical sensor, the fiber end facet structures may also be added to the other fiber optical sensors described herein to further enhance acoustic response. For example, a metasurface may include patterns of small elements arranged so as to change the wavefront shape of the acoustic signals and maximize the collection of acoustic signals collected by the other types of fiber optical sensors discussed herein to improve the sensitivity of the fiber optical sensors. Adding low-dimensional materials to a fiber end facet may also improve sensitivity because such materials are more prone to deformation induced by acoustic waves, which may translate into larger changes in the optical signal. By writing plasmonic patterns onto a fiber end facet, it is possible to enhance the optical response to acoustic waves. This enhancement may be achieved through leveraging the hotspots and resonances generated by these plasmonic patterns to amplify light-matter interactions. As used herein, “low-dimensional” or “2 dimensional” features may refer to features having a thickness of less than 1 micron.

The aforementioned optical structures are configured to respond to acoustic (such as ultrasound) signals. Thus, these optical structures may include acoustically responsive materials and/or acoustically responsive structures. Acoustically responsive, as used herein, refers to structures or materials that are configured to respond to incident acoustic signals (e.g., ultrasound acoustic signals) in a manner that adjusts the optical properties of the materials or structures. Reponses to acoustic signals in such resonant, interferometer or acoustically responsive fiber end facet structures may be due to the photo-elastic effect and/or physical deformation of the structures. When subject to acoustic signals, the resonant, interferometer or acoustically responsive fiber end facet structures are subject to mechanical stress and/or strain from the alternating pressures of the acoustic signal sound waves. This mechanical stress and/or strain may change the optical properties of the optical sensor structures due to the photo-elastic effect and may also cause changes or deformations in the physical structure of resonator. With polarization-based sensors, the polarization of optical signals changes when the medium through which the light is passing is subjected to acoustic signals. When coupled to a light source (e.g. a laser light source, a broadband light source (e.g. a lamp or LED) or other suitable light source) via an optical waveguide (e.g., an optical fiber), the effect of acoustic signals on the optical sensor structures may be measured due to changes in the light returned by the optical sensor structures via the optical waveguide. Within this disclosure, optical signals and light may be referred to as responding to acoustic signals. It is understood that such responses are due to the interaction between the acoustic signals and the medium through which the light passes. Thus, as discussed herein, a material or structure that is referred to as acoustically responsive may respond to acoustic signals typical of an ultrasound environment in manner that can be measured, by techniques discussed herein, by optical signals consistent with embodiments hereof.

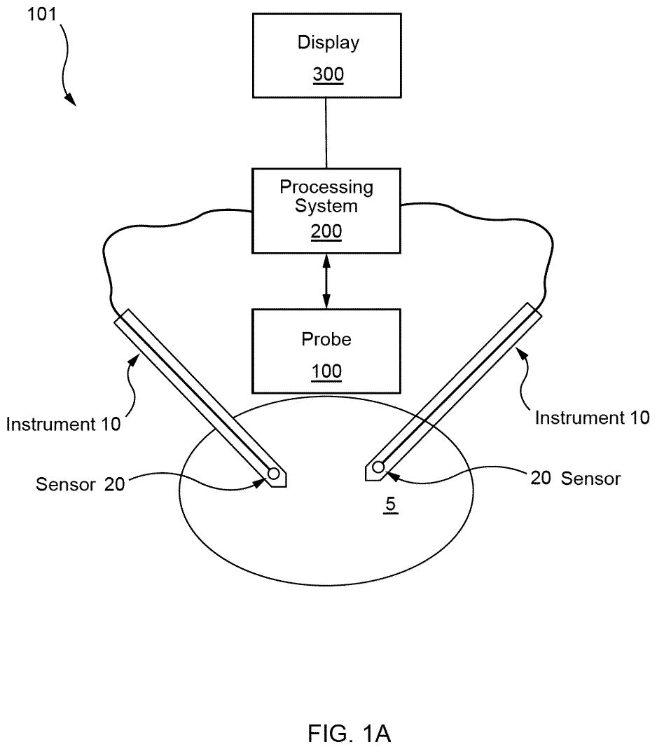

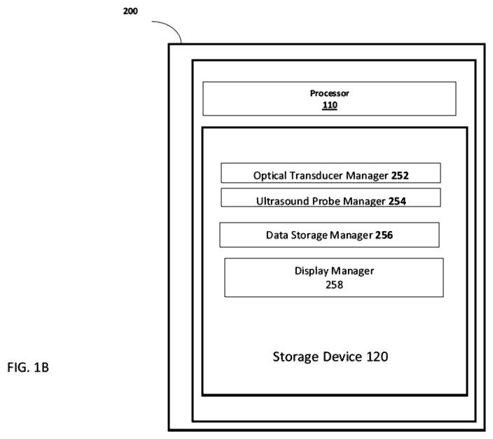

Turning to FIG. 1A, FIG. 1A is an example of a medical guidance system 101 for ultrasound visualization of a transponder or beacon, such as an optical transducer coupled to a medical device, such as a surgical tool. Medical guidance system 101 may be used for ultrasound transponder visualization, location, monitoring, and guidance of a medical device, such as instrument 10, which may be, as illustrated, a needle, present in a medium 5 (e.g., body tissue, body cavity, body lumen). However, it should be understood that in other examples the medical guidance system 101 may be used for ultrasound visualization of other medical devices and surgical instruments such as, but not limited to a catheter, an introducer, a snare, a guidewire, an intravenous (IV) line, an endoscope, a trocar, an implant, an endoscope, blunt dissection tools, combinations thereof, and any other surgical, diagnostic, or medical instrument. Medical guidance system 101 may also be used to enhance visualization of aspects present in the medium 5, such as, for example, organs, vessels, tissue, tumors, other anatomical structures, other medical devices, or implants. Medical guidance system 101 may further be used to generate an ultrasound image from the location of the sensor-which can be used to enhance the ultrasound image or generate an additional image from the perspective of the sensor or sensors.

In some examples, the medical guidance system 101 may include a medical guidance processing system 200 in communication with an ultrasound probe 100, an optical transducer 20 coupled to a medical device such as instrument 10, and a display 300. The optical transducer 20 is an on-tool ultrasound based optical sensor. The optical transducer 20 may further be referred to as a “transponder sensor,” “beacon sensor,” “optical sensor,” etc. Such descriptions do not limit the optical transducer 20 to any functionality associated with such terms. In some examples, the instrument 10 may include more than one optical transducer 20 or combinations of optical transducers 20. While the optical transducer 20 is shown in FIG. 1A as a single element, there may be separate multiple elements arranged adjacent or spaced apart from each other or in an array form factor. During a procedure, the instrument 10 may be inserted into the medium 5. The optical transducer 20 (e.g., coupled with instrument 10) is arranged to be moved independently from motion of the probe 100.

The transducer/probe 100 may emit acoustic beamforming pulses or signals and/or acoustic beacon pulses or signals toward a medium 5. In some variations, the medium 5 may be a non-linear medium, for example, a body tissue. An optical transducer 20 arranged on at least a part of the instrument 10 may detect acoustic beamforming and/or acoustic beacon signals. The optical transducer 20 may convert the received acoustic signals into optical signals. The optical signals may be transmitted to the medical guidance processing system 200 via an optical fiber or other suitable waveguide where they are converted to an electrical signal, e.g., after receipt by a photodetector. The present disclosure may refer to the medical guidance processing system 200 receiving, interpreting, analyzing, etc. the received acoustic beamforming and/or acoustic beacon signals. In such instances, it is understood that that the medical guidance processing system 200 receives electrical signals, such as from a photodetector, corresponding to the optical signals caused by interaction of the optical transducer 20 with the acoustic beacon and acoustic beamforming signals.

The ultrasound probe 100 may detect acoustic beamforming signals reflected in response to interactions of the acoustic beamforming pulses or signals with the medium 5 and/or the instrument 10. The ultrasound probe 100 may transmit the detected acoustic beamforming signals to the medical guidance processing system 200. The medical guidance processing system 200 may generate ultrasound images from the ultrasound probe's detected acoustic beamforming signals and/or the optical transducer 20 detected acoustic signals. The medical guidance processing system 200 may also analyze the optical sensors sensed acoustic beamforming and/or acoustic beacon signals to generate an object indicator to determine a location of the sensor, and hence the needle within the ultrasound image. The ultrasound images and the object indicator may be displayed on a display 300.

Although the instrument 10 in FIG. 1A is shown to be a needle, it should be readily understood that any suitable object may be visualized and/or tracked using the medical guidance system 101. For example, the medical guidance system 101 may be used to visualize and/or track a catheter as it is being advanced into a blood vessel, body lumen, body tissue, and/or organ. Any other medical device, surgical tool or instrument, or other object may be similarly tracked via the inclusion of an optical transducer 20 as discussed herein.

FIG. 1B illustrates a medical guidance processing system 200 consistent with embodiments hereof. The medical guidance processing system 200 includes one or more processors 110 and one or more storage devices 120.

The medical guidance processing system 200, may be configured to transmit electrical signals to excite the elements in the ultrasound probe 100. Additionally, the medical guidance processing system 200 may receive electrical signals that are a representation of converted ultrasound echoes (e.g., beamforming signals) from the ultrasound probe 100. The medical guidance processing system 200 may process these electrical signals to generate ultrasound images. The medical guidance processing system 200 may also receive converted optical signals that may be a representation of beacon signals and/or beamforming signals received by the optical transducer 20. The processing system 200 may process these converted optical signals to generate object or location indicators and/or to determine a location of an object (e.g., instrument 10). The medical guidance processing system 200 may further process the converted optical signals to generate ultrasound images to enhance the image generated by the converted ultrasound echoes or to create an ultrasound image from the perspective of the sensor.

The processor 110 is programmed by one or more computer program instructions stored on the storage device 120. For example, the processor 110 is programmed by an optical transducer manager 252, an ultrasound probe manager 254, a user interface manager 255, a data storage manager 256, and a display manager 258. It will be understood that the functionality of the various managers as discussed herein is representative and not limiting. Additionally, the storage device 120 may act as a data retention system to provide data storage. As used herein, for convenience, the various “managers” will be described as performing operations, when, in fact, the managers program the processor 110 (and therefore the medical guidance processing system 200) perform the operation.

The various components of the medical guidance medical guidance system 101 work in concert to provide integrated guidance, tracking, location, and visualization of one or more surgical or interventional tools. Such integration may include, as needed, exchange of information, data, and control signals with all components of the medical guidance system 101. In embodiments, as discussed above, the various components of the medical guidance system, including the surgical processing system 200, the display 300, the surgical instruments 10, and the ultrasound probe 100 may be provided as a single unified and integrated system. In further embodiments, any or all aspects of the medical guidance system 101 may be provided as separate individual components integrated into the medical guidance system 101 through communication via the medical guidance processing system 200.

The physical hardware of the medical guidance system 101 may be instantiated in various ways across these systems. Further, the functionality and control of these systems may also be instantiated in different ways across these systems. Thus, the arrangement of managers described herein is provided by way of example, and the various managers may be operational on different hardware associated with different systems without limitation, depending on the particular embodiment.

In one example embodiment, the medical guidance processing system 200 may be provided in an integrated unit with the display 300 and connections for the ultrasound probe 100 and surgical instruments 10. In other embodiments, one or more of the display 300, the ultrasound probe 100, and the surgical instruments 10 may be connected to and controlled by different systems that are in communication with the medical guidance processing system 200. For example, the surgical instruments 10 may be connected to and controlled by an ablation system and/or the ultrasound probe 100 may be connected to and controlled by an ultrasound system. In such embodiments, the necessary communication connections may be provided to medical guidance processing system 200 to receive signals acquired by these devices.

The optical transducer manager 252 is a software protocol operating on the medical guidance processing system 200. The optical transducer manager 252 is configured to manage communications with one or more optical transducers 20 associated with the surgical instruments 10. The optical transducer manager 252 may control the optical (e.g., laser) signals provided to the optical transducers 20 and may be configured to receive signals associated with or corresponding to the optical signals from the optical transducers 20. Thus, the optical transducer manager 252 may control a light source configured to provide an optical signal to the optical transducers 20 and may be in communication to receive signals from a light reception device, such as a photodetector, that correspond to optical signals returned from the optical transducers 20. In embodiments, the signals from the light reception device may be location signals that correspond to optical signals returned from the optical transducers in response to beacon signals provided by the ultrasound probe 100. In embodiments, the signals from the light reception device may be imaging signals that correspond to optical signals returned from the optical transducers in response to beamforming signals provided by the ultrasound probe 100. Accordingly, the optical transducer manager 252 is configured to obtain location signals that correspond to optical signals returned from the optical transducers 20 in response to received beacon signals and is configured to obtain imaging signals that correspond to optical signals returned from the optical transducers 20 in response to received beamforming signals.

The ultrasound probe manager 254 is a software protocol operating on the medical guidance processing system 200. The ultrasound probe manager 254 is configured to provide control and communication with the necessary hardware to operate the ultrasound transducer probe 100. In embodiments that include an externally connected ultrasound system, the ultrasound probe manager 254 is configured to provide communication with the ultrasound system. The ultrasound probe manager 254 is configured to cause the generation of beacon signals and beamforming signals by the ultrasound transducer probe 100. The ultrasound probe manager 254 is further configured to communicate with the optical transducer manager 252 to translate the received location signals from the optical transducers 20 into a location of each optical transducer 20 (and therefore the surgical instruments 10 on which they are disposed). The ultrasound probe manager 254 is further configured to communicate with the optical transducer manager 252 to translate or interpret received imaging signals from the optical transducers 20 to generate ultrasound imagery from the point-of-view of the optical transducers 20.

The data storage manager 256 is a software protocol operating on the medical guidance processing system 200. The data storage manager 256 is configured to access the one or more data retention systems 190 and/or the storage device 120 to store any and all data associated with embodiments herein.

The display manager 258 is a software protocol operating on the medical guidance processing system 200. The display manager 258 is configured to provide a user interface, e.g., a graphical user interface provided via the display 300, to provide information and allow user interaction with the medical guidance system 101 and the medical guidance processing system 200. The display manager 258 is configured to receive input from any user input source, including but not limited to touchscreens, keyboards, mice, controllers, joysticks, voice control. The display manager 258 is configured to provide a user interface, such as a text based user interface, a graphical user interface, or any other suitable user interface. The display manager 258 may be configured to provide different user interface services depending on a type of client device. For example, a laptop or desktop computer or ultrasound console display screen may be provided with a user interface including a full suite of interface options, while a smartphone or tablet may be provided with a user interface limited to status updates. In embodiments, the display manager 258 may be configured to provide or cause the display of a graphical user interface (GUI), as described below. It is understood that any information that the display manager 258 is configured to cause the display of may be computed, determined, and/or otherwise obtained by the medical guidance processing system 200.

The output from the processing system 200 may be sent to the display 300, as directed by the display manager 258. The display 300 may be operatively coupled to the medical guidance processing system 200 and may show ultrasound images (e.g., real-time ultrasound images) and object or location indicators (e.g., graphic or other icon, trace, grid, and/or other visual indicators) that may be representative of a position of an object. In some variations, the display 300 may show the ultrasound images and the object indicators in real time. In some variations, the object indicators may be overlayed with the ultrasound images. For instance, the ultrasound images may be displayed on the display 300 and the object indicators may be displayed over the ultrasound images on the display 300. Object or location indicators may include any suitable visual indicator representative of the position of the object (e.g., instrument 10). For example, object indicators may be a graphic that is positioned over an anatomical image, such as an ultrasound image, to represent the current position of an instrument 10 relative to other instruments 10 and/or other objects (e.g., tissue features) in the anatomical image. As such, the location of the object indicator(s) may communicate position within a field of view of the ultrasound probe 100.

The display manager 258 may be configured to obtain and cause display of one or more location indicators corresponding to locations of the surgical instruments 10. As discussed herein, the location of the surgical instruments 10 may be determined according to the receipt of beacon signals generated by the ultrasound probe 100 and received by the optical transducers. The location indicators, in some embodiments, may be displayed on an anatomical map. The anatomical map may be obtained, for example, by any suitable imaging technology, including CT, fluoroscopy, ultrasound (e.g., generated according to beamforming signals from the ultrasound probe 100 and/or via a separate ultrasound probe/system), etc.

In embodiments, the display manager 258 may be configured to obtain and display orientation information of the one or more instruments 10. For example, in embodiments that include multiple optical transducers 20 located on a single instrument 10, the orientation of the instrument 10 may be readily determined. In the case of a needle, for example, two optical transducers 20 may provide information about the direction the needle extends in. In the case of a catheter, in another example, multiple optical transducers 20 may provide suitable information about the location of various portions of the catheter, thereby providing information about the overall shape and orientation of the catheter.

In embodiments, the display manager 258 may be configured to obtain and display an in-plane indicator corresponding to one or more of the surgical instruments 10. Conventional ultrasound may generate a planar image with a relatively low elevation thickness. Objects outside of the image plane may still be within an insonified region and thus may still receive and react to ultrasound signals. Due to the nature of two-dimensional displays displaying planar images, it may be difficult to discern whether an object in an insonified region is also within an imaging plane. To assist operators, an in-plane indicator may be displayed in conjunction with the location indicator. Such an in-plane indicator may include additional markings, such as hash marks, and/or different colorations, such as a color transition from red to green, to indicate whether an instrument 10 is within or outside of the imaging plane and how close it may be to the imaging plane. Although referred to as an “in-plane” indicator, the in-plane indicator of the present disclosure may be configured to indicate either or both of in-plane and out-of-plane status. In some embodiments, the in-plane indicator may be configured to disappear when in-plane status is achieved. In some embodiments, the in-plane indicator may be configured to appear when in-plane status is achieved. In some embodiments, the in-plane indicator may be configured to change depending on how far the located object is from the imaging plane.

In embodiments, the display manager 258 may be configured to obtain and display a rendezvous guide to assist an operator with a rendezvous procedure between a first instrument 10 and a second instrument 10. Such a rendezvous guide may include, for example, imagery of the path or track of the instrument 10 in relation to the anatomical map and/or may include a predicted or projected pathway of the instrument 10 should it continue along the established path.

In embodiments, the display manager 258 may be configured to obtain and display location indicators corresponding to the surgical instruments 10 such that there location is provided relative to one another. For example, the medical guidance processing system 200 may accurately determine the spatial relationship between one or more surgical instruments 10 according to the receipt of the beacon signals. Such may be possible because the one or more surgical instruments are receiving beacon signals from the same ultrasound probe 100. When displayed, the spatial relationship between the one or more surgical instruments 10 may be provided such that the relative locations between the surgical instruments 10 is maintained. Accordingly, xyz coordinates of the surgical instruments 10 may be established such that the relative locations of the surgical instruments 10 are determined from the beacon signals. In embodiments, the xyz coordinates may be displayed to assist the operator. In embodiments that include anatomical imagery, it may be necessary to register the anatomical imagery with the identified locations of the surgical instruments 10. For example, if the anatomical imagery is produced by an alternative system (e.g., CT, etc.), the medical guidance processing system 200 may be configured to register the identified locations of the surgical instruments 10 with the anatomical imagery.

In embodiments, the display manager 258 may be configured to obtain and display location indicators corresponding to the surgical instruments 10 such that their location is provided relative to an external framework. For example, in an embodiment, the ultrasound probe 100 may be employed to generate an ultrasound image for display. The generated ultrasound image for display may represent an external framework. Because the spatial relationship of the origin of acoustic signals to generate the ultrasound image and to locate the surgical instruments 10 is known, the xyz coordinates of the surgical instruments 10 within the ultrasound image may be established by the medical guidance processing system 200. n embodiments, the xyz coordinates may be displayed to assist the operator. Accordingly, the locations of the surgical instruments 10 may be provided to the display relative to the ultrasound image without a need for additional registration between the instruments and the image. In such embodiments, the locations of the surgical instruments may further be provided with respect to one another.

In embodiments, the display manager 258 may be configured to obtain and cause display of location indicators, orientation indicators, in-plane indicators, and any other relevant information in real-time or substantially real-time. By “substantially real-time” it is meant that the imagery is updated continuously and quickly enough to provide feedback to an operator on the current location of the surgical instruments 10. The physical systems involved may introduce small or insignificant timing delays without departing from a substantially real time display.

In embodiments, the display manager 258 may be configured to obtain and cause display of instrument path history. As discussed above, the medical guidance system 200 may obtain location information associated with the instruments 10. This location information may be displayed in the form of location indicators in real-time. The location information may also be stored for subsequent display in non-real-time to indicate the locations that the instruments 10 have previously occupied. Such information may be provided in the form of historical location indicators which may be used, for example, to indicate a previous location the instruments 10 at specific times (for example, while a specific procedure was conducted). Such information may also be provided in the form of a path history indicator, e.g., to indicate a path that that instrument 10 has traveled through to arrive at a preset location. Such historical path information may be used to generate a trajectory of the instruments 10, which may in turn be used to generate a predicted future path of the instruments 10, e.g., should they continue along a same trajectory, displayed as a trajectory indicator. Path history indicators and trajectory indicators may be visualized as lines or a series of markers indicating the historic path or predicted future trajectory of an instrument 10.

The optical transducer 20, e.g., the transponder or beacon, may include an optical sensor configured as an on-tool ultrasound based optical sensor, such as an interferometer sensor, a resonator sensor, a sensor including Fiber Bragg gratings, a fiber end facet with acoustic responsive structures, and/or a polarization (birefringence) sensor (e.g., as disclosed in U.S. Pat. No. 12,025,489, titled “FIBER-OPTICAL SENSOR SYSTEM FOR ULTRASOUND SENSING AND IMAGING,” issued on Jul. 2, 2024, the contents of which are hereby incorporated by reference. The optical sensor may be located on a tool (e.g., on-tool), medical device surgical instrument, etc. The optical sensor may be configured to receive acoustic signals, e.g., acoustic beamforming signals and acoustic beacon signals. The fiber end facet structures may include acoustically responsive microstructures, such as metasurfaces including patterns of small elements arranged to change the wavefront shape of the acoustic signals and maximize the detection of acoustic signals, acoustically responsive low-dimensional materials with optomechanical features selected to optimize acoustic response (e.g., features that are more prone to deformation when receiving acoustic signals, exhibit greater material responses to acoustic signals) and plasmonic structures patterned to amplify light-matter interactions. Plasmonic structures may locally amplify incident light due to their plasmonic resonance. The transponder or beacon may be used to locate a device's location and/or orientation while a fiber sensor is mounted on the device based on incident acoustic beacon signals. The device may be a needle, catheter, introducer, endoscope, surgical tool, biopsy tool, blunt dissection tool, stylet, and any other surgical, diagnostic, or medical instrument.

The example optical transducer 20 may include an ultrasound based optical sensor, such as a point sensor, a line sensor, or a sensor formed in some other known shape. The optical transducer 20 may be coupled to one end of the instrument 10, such as the distal end of a needle, e.g., the end that first penetrates the tissue or enters a body cavity or lumen. In some examples, multiple transponders may be coupled to the instrument 10. For instance, one transponder may be coupled to the distal end while another is coupled to the mid-point of the needle or other area that will provide positional information helpful during the procedure. The optical transducers 20 may be disposed at the end of an optical fiber, adjacent an end of an optical fiber or at a diagnostic or therapeutically relevant location to create an appropriate sensor for the medical device.

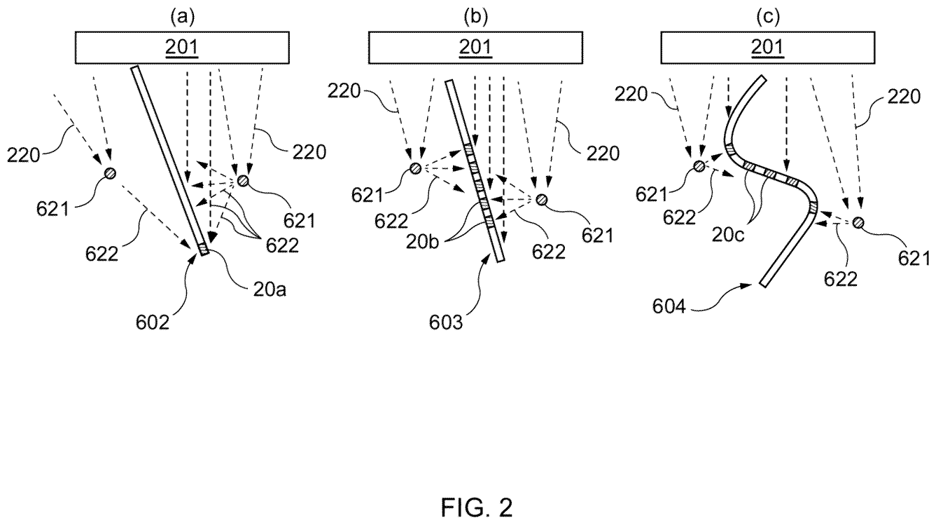

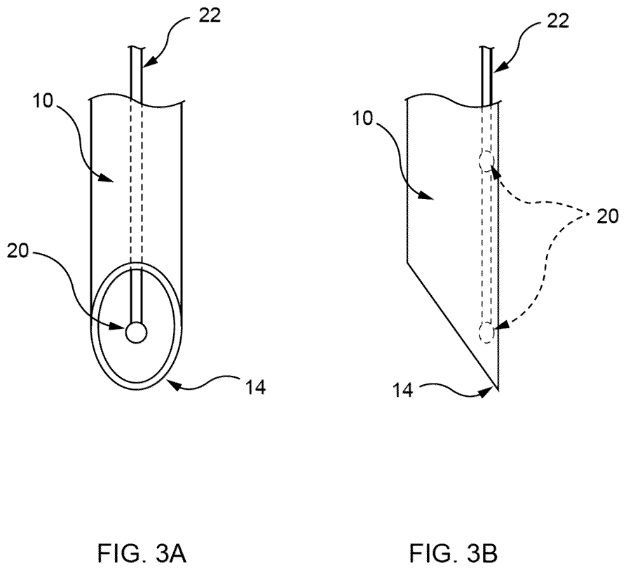

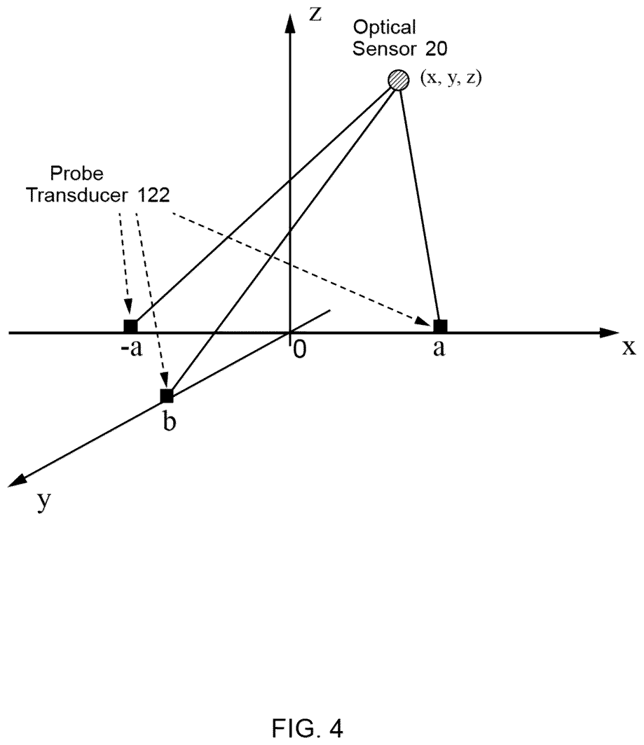

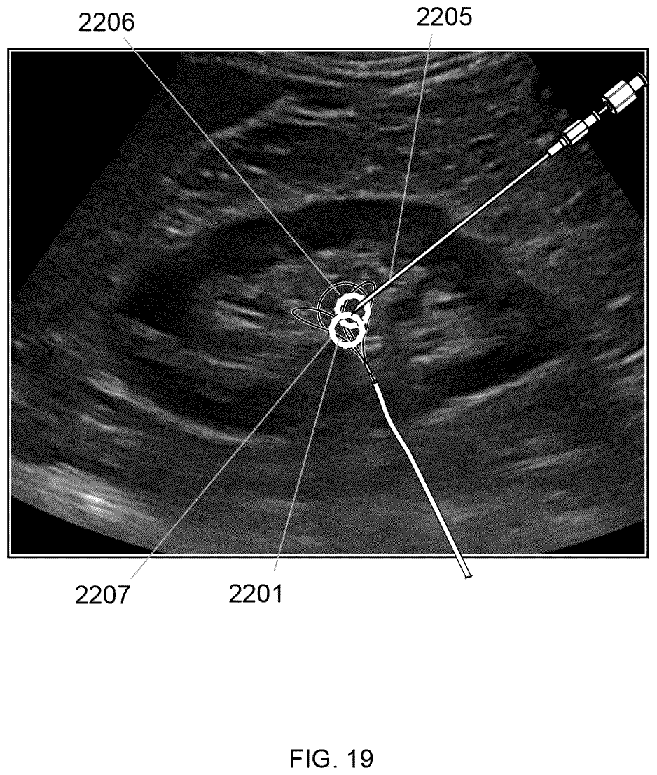

Optical transponder sensors (e.g., optical transducer 20 in FIG. 1 or 2A) may be “point like” in that the optical transducer 20 has a dimension close to or smaller than a certain feature size that is meaningful for an application, such as a wavelength of an acoustic signal or a diameter of a needle (e.g., optical transducer 20a in FIG. 3A). The point like sensor can receive acoustic signals transmitted from an ultrasound probe or scattered signals from surrounding tissue regardless of its orientation to the signals it receives. The point like sensor may be configured to detect acoustic signals across a directional range of at least 180 degrees, at least 270 degrees, at least 300 degrees, or at least 330 degrees. As used herein, the directional range refers to a range in the plane of the optical transducer 20. As discussed above, the optical transducer 20 may be disposed at the end of an optical fiber. The plane of the optical transducer 20 refers to the plane that an optical fiber extending directly from the optical transducer 20 with no bending would occupy. In some embodiments, the sensor may be configured to detect acoustic signals in an omni-directional or omni-receptive fashion, e.g., across a range of 360 degrees. Optical transducers 20 consistent with embodiments hereof may also be “line-type) sensors. Line type sensors may extend over a defined length and may be configured to receive incident acoustic signals laterally. Line type sensors may be single sensors extended over the length of the line type sensor and/or may include several discreet sensors located in-line with one another to form the line geometry. A line type sensor may use a polarization sensitive detection mechanism in an optical fiber disposed within an insonified region (e.g., during a medical procedure). A line type fiber sensor may be straight, e.g., as in optical transducer 20b shown in FIG. 2B, or be arranged in a shape, e.g., as in optical transducer 20c shown in FIG. 20c, to facilitate different applications. For example, a line type optical transducer 20 may be curved to form a “focused” type sensor that (e.g., optimally) detects ultrasound coming from a designed focusing spot or to conform to the shape of the medical tool/device. It is also to be understood that a line-like optical transducer 20 is not limited to a fiber sensor using birefringence (e.g., a polarization sensitive detection mechanism), as multiple point like optical sensors may be arranged to form a line like arrangement.

In embodiments, optical transducers 20 including optical fiber based sensors, both point-like and line-type, as described herein may be formed to include or may be formed adjacent to acoustic enhancement materials configured to increase the sensitivity of the optical sensors and/or to improve impedance matching with surrounding tissue.

In embodiments, the medical guidance processing system 200 may construct or generate an image based solely on the optical signals received from one or more optical transducers 20 in response to receipt of acoustic signals, e.g., to create an image from the perspective of the one or more optical transducers 20. In embodiments, the optical signals received from one or more optical transducers 20 may be used in conjunction with the acoustic signals received by a traditional ultrasound probe, e.g., to enhance an image created from the perspective of the traditional ultrasound probe.

These principles are illustrated in greater detail in FIG. 2(a)-2(c) As shown in FIG. 2(a)-2(c), an acoustic probe 201 (e.g., such as an ultrasound probe 100) may be used to transmit acoustic signals 220 (e.g., acoustic beamforming signals) into an area of interest. The acoustic probe 201 may function as a traditional acoustic probe to detect reflections of the acoustic signals 220 for imaging purposes. These images may be enhanced by additional information obtained by one or more on-tool ultrasound transducers 20. The acoustic signals 622 may result from reflection, scattering, and/or tissue harmonics. As shown in FIG. 2(a)-2(c), the acoustic signals 622 may be generated from the interaction of the acoustic signals 220 with points 621 within the area of interest.

The optical transducers 20a of sensor fiber 602 may be configured to receive acoustic signals 622 from any direction, as discussed herein. The sensor fiber 603 may be configured with a plurality of polarization based optical sensors 20b, as discussed herein, and may receive acoustic signals 622 from directions lateral to the axis of the sensor fiber 603. As used herein, “lateral” refers to all directions that are not parallel to the axis of the sensor fiber 603. As shown in FIG. 2B, the acoustic signals 622 may be received by the sensor fiber 603 at any exposed portion along its length and from any direction (each exposed portion acting as a optical transducer 20b). Further the polarization of the sensor fiber 603 may be selected or adjusted to accommodate an expected or desired radial angle of incidence of the acoustic signals 622. FIG. 2C further illustrates the sensor fiber 604, which may curve within the area of imaging interest. Similar to the sensor fiber 603, the sensor fiber 604 may be configured with a plurality of polarization based optical sensors 20c to detect incident acoustic signals 622 received from a lateral, substantially lateral, or from any direction relative to the axis of the sensor fiber 604. Detection of lateral signals at multiple points along the length of the sensor fiber 604 may enhance an ability to track and/or locate the sensor fiber 604 when it is disposed within a medium (e.g., within a human body during a medical procedure). For example multiple signals incident along the length of the sensor fiber 604 may enhance an ability to determine the location of different portions of the sensor fiber 604 along its length and therefore to identify the location of the entire sensor fiber 604, and not just a tip region. For example multiple signals incident along the length of the sensor fiber 604 may enhance an ability to determine the location of different portions of the sensor fiber 604 and therefore to identify curvature of the sensor fiber 604 with greater accuracy.

FIGS. 2A, 2B, and 2C depict embodiments of sensing using sensors 20a, 20b, and 20c. In FIG. 2A, optical transducer 20a, a point-like sensor, is a fiber based optical sensor that can receive acoustic signals, including scattered acoustic signals from any direction. In FIG. 2B, sensors 20b are fiber polarimetric optical sensors that form a straight line receiver. In some embodiments, a single long optical transducer 20b may be used. Optical transducer 20b receives acoustic signals, including scattered acoustic signals from lateral directions. In FIG. 2C, sensors 20c are fiber polarimetric sensors that form a curved line receiver. In some embodiments, a single long curved line optical transducer 20c may be used. Optical transducer 20c receives acoustic signals, including scattered acoustic signals, from lateral directions. Accordingly, the optical sensor structures, as shown in FIGS. 2A, 2B, and 2C, are configured to detect the acoustic signal across a directional range of at least 180 degrees, at least 270 degrees, at least 300 degrees, at least 330 degrees, or at least 360 degrees.

Detection of lateral signals at multiple points along the length of the sensors 20b and 20c may enhance an ability to track and/or locate the sensor fibers when it is disposed within a medium (e.g., within a human body during a medical procedure). For example, as shown in FIGS. 2B and 2C, multiple signals incident along the length of the sensor fibers may enhance an ability to determine the location of different portions of the sensor fibers along its length and therefore to identify the location of the entire sensor fibers 20b and 20c, and not just a tip region like 20a. For example, as shown in FIGS. 2B and 2C, multiple signals incident along the length of the sensor fibers 20b and 20c may enhance an ability to determine the location of different portions of the sensor fibers 20b and 20c and therefore to identify curvature of the sensor fibers 20c with greater accuracy.

Referring again to FIG. 1A, the ultrasound probe 100 may include an ultrasound array with one or more elements (e.g., transducers) to generate acoustic pulses and/or receive acoustic signals (e.g., echo signals) corresponding to the acoustic pulses. For example, the ultrasound array may include one or more elements (e.g., transducers) configured to emit a set of acoustic beamforming pulses (e.g., ultrasound signals) and/or receive a set of acoustic beamforming signals (e.g., ultrasound echoes) corresponding to the set of acoustic beamforming pulses. Furthermore, the ultrasound probe 100 may also include one or more elements (e.g., transducers) to emit acoustic beacon pulses. The beamforming signals that correspond to beamforming pulses may be used to generate ultrasound images. Beacon signals that correspond to beacon pulses may be used for object tracking. In some variations there is no need for specific elements dedicated to emitting acoustic beacon pulses, e.g., emitted acoustic beamforming signals may function as acoustic beacon signals. For example, as discussed above, beacon signals may be converted into optical signals by the optical transducers 20 that may be analyzed to determine a location of the object and/or to generate an object indicator, analyzed to enhance the ultrasound image, and/or create an ultrasound image from the perspective of the sensor.

In some examples, the elements of the ultrasound probe 100 may be arranged as an ultrasound array. For example, the ultrasound probe 100 may include one or more acoustic energy generating (AEG) transducers, such as one or more of a piezoelectric transducer, a lead zirconate titanate (PZT) transducer, a polymer thick film (PTF) transducer, a polyvinylidene fluoride (PVDF) transducer, a capacitive micromachined ultrasound transducer (CMUT), a piezoelectric micromachined ultrasound transducer (PMUT), a photoacoustic transducer, a transducer based on single crystal materials (e.g., LiNb03(LN), Pb(Mg113Nb213)-PbTiQ3 (PMN-PT), and Pb(In112Nb112)-Pb(Mg113Nb213)PbTiQ3(PIN-PMN-PT)), combinations thereof, and the like. It should be understood that the probe 100 may include a plurality of any of the transducer types. In some examples, the ultrasound array may include the same type of elements. Alternatively, the ultrasound array may include different types of elements. The ultrasound probe 100 can be a traditional ultrasound probe with an acoustic energy generating transmitter and receiver, and/or the ultrasound probe 100 can be an acoustic-optical probe that includes acoustic energy generating transmitters and optical based acoustic receivers (e.g., as described in U.S. application Ser. No. 17/990,596, filed on Nov. 18, 2022 titled “Mixed Ultrasound Transducer Arrays,” US application Ser. No. 17/244,605 filed on Apr. 29, 2021 titled “Modularized Acoustic Probe,” U.S. application Ser. No. 18/597,493 filed on Mar. 6, 2024 and titled “Mixed Array Imaging Probe,” and U.S. application Ser. No. 19/047,479 titled “Photonic Integrated Acoustic Sensor,” each of which is hereby incorporated by reference). In some examples that include the acoustic-optical probe, an ultrasound array may include one or more optical sensors, such as an interference-based optical sensor, which may be one or more of an optical interferometer, optical cavity, optical resonator (e.g., whispering gallery mode (WGM) resonators among others), birefringent sensor, or an optical fiber end facet with an acoustic-responsive structure.

FIG. 3A illustrates an example of a system in which an optical transducer 20 is attached to an instrument 10, depicted here as a needle, at one end to facilitate it's tracking and position determination. In FIG. 3A, the optical transducer 20 may be attached to, coupled to, integrated with, or otherwise mounted on a distal end (e.g., a tip or stylet) of the instrument 10.

The optical transducer 20 may be arranged on (e.g., coupled to, mounted on, integrated with, or otherwise located on) the instrument 10 in any suitable manner, such as with epoxy or mechanical interfit features. FIG. 3A illustrates an exemplary variation of a system in which an optical transducer 20 is attached to an instrument 10 to track the instrument 10 and/or determine a position of the instrument 10. In FIG. 3A, the optical transducer 20 may be attached to, coupled to, integrated with, or otherwise mounted on or near a distal end of the instrument 10. The optical transducer 20 may detect acoustic beacon and/or acoustic beamforming signals reflected in response to interactions of the acoustic beacon pulses and/or acoustic beamforming signals from a probe (e.g., probe 100 in FIG. 1) with the distal end of the instrument 10. The optical transducer 20 may detect the acoustic beacon and/or acoustic beamforming signals through photo-elastic effect and/or physical deformation of the optical transducer 20. For example, in the presence of acoustic signals, light and/or sound waves (e.g., WGMs) traveling the optical transducer 20 may undergo a spectral shift caused by changes in the refractive index and shape of the optical transducer 20. The optical transducer 20 may transmit optical signals representative of the detected acoustic beacon and/or acoustic beamforming signals to a processing system (e.g., processing system 200 in FIG. 1). The optical transducer 20 may be coupled to one or more optical waveguides 22 (e.g., optical fibers, photonic integrated circuit waveguides, and/or the like) to propagate the optical signals to the processing system. The medical guidance processing system 200 may generate an object or location indicator from the optical signals. The object or location indicator may be representative of a position of the distal end of the instrument 10 and/or may track the distal end of the instrument 10. For example, the distal end of the instrument 10 may be visualized and tracked based on the object indicator. Accordingly, an instrument 10 may be reliably visualized and tracked during a medical procedure using a single optical transducer 20 by visualizing and tracking the distal end of the instrument 10.

In embodiments, the instrument 10 may be an introducer, such as an axial introducer sheath or other type of introducer. Such introducers may be employed to assist in guiding or introducing another instrument, such as a needle, ablation catheter, etc. to an appropriate treatment site. In embodiments, such an introducer may include an optical transducer 20 as discussed herein. In embodiments, both an introducer and another instrument guided by the introducer may be instruments 10 and each may have an optical transducer 20 disposed thereon.

FIG. 3B illustrates an exemplary variation of a system in which two optical sensors 20 are attached to an instrument 10 (depicted here as a needle) to track the instrument 10 and/or determine a position of the instrument 10. As seen in FIG. 2B, one optical transducer 20 may be arranged on a distal end of the instrument 10 while another optical transducer 20 may be arranged on an elongate member of the instrument 10. Accordingly, the optical transducer 20 on the distal end of the instrument 10 and the optical transducer 20 on the elongate member of the needle may detect acoustic beacon and/or acoustic beamforming signals generated by the ultrasound probe 100. Both the optical transducers 20 (e.g., the optical transducer 20 on the distal end and optical transducer 20 on the elongate member) may be coupled to a same waveguide 22 or to a different waveguide 22 (e.g., optical fibers, photonic integrated circuit waveguides, and/or the like) to propagate the optical signals to a processing system (e.g., medical guidance processing system 200 in FIG. 1). The medical guidance processing system 200 may generate a first location indicator representative of a position of the distal end of the instrument 10 based on the optical signals from the optical transducer 20 at the distal end of the instrument 10 and a second location indicator representative of a position of the elongate member of the instrument 10 based on the optical signals from the optical transducer 20 at the elongate member of the instrument 10. Alternatively, the processing system may generate a single object indicator that may represent both a position of the distal end of the instrument 10 based on the optical signals from the optical transducer 20 at the distal end of the instrument 10 and a position of the elongate member of the instrument 10 based on the optical signals from the optical transducer 20 at the elongate member of the instrument 10. Therefore, an instrument 10 may be reliably visualized and tracked during a medical procedure by visualizing and tracking both the distal end of the instrument 10 and the elongate member of the instrument 10.

Although FIG. 3A illustrates a single optical transducer 20 for visualizing and tracking an instrument 10 and FIG. 2B illustrates two optical transducers 20 for visualizing and tracking an instrument 10, it should be readily understood that any suitable number of optical sensors may be used to visualize and track the needle (e.g., three or more optical sensors, such as three, four, five, or more optical sensors). These optical sensors may be attached to, coupled to, integrated with, or otherwise mounted on any suitable part of the instrument 10. For example, using three optical transducers 20 on a single instrument 10 (e.g., one at the distal end, and two along the elongate member of the needle) may accurately track a bending of the instrument 10 in addition to visualizing and tracking the position of the distal end. medical guidance. It should be readily understood that any suitable object (e.g., catheter, guidewire, endoscope, trocar, implant, and any other surgical, diagnostic, or medical instrument) may be visualized and/or tracked using the systems and methods described herein.

As discussed above, information captured from receipt of the acoustic beacon and/or acoustic beamforming signals by the optical transducer 20 may also be used to enhance an ultrasound image generated by another transducer (e.g., and external probe), and/or to generate an ultrasound image from the perspective of the optical transducer 20.

Further examples of optical sensors and transducers that may be used for visualizing and/or tracking a surgical tool may include fiber based sensors, such as those described in U.S. Pat. No. 12,025,489, titled “FIBER-OPTICAL SENSOR SYSTEM FOR ULTRASOUND SENSING AND IMAGING,” issued on Jul. 2, 2024 and Ser. No. 18/609,378 , titled “FIBER-OPTICAL SENSOR ARRAY FOR SENSING AND IMAGING,” filed on Mar. 19, 2024, each of which is incorporated by reference in its entirety.

Depending upon the medical application, the instrument may change its orientation with respect to the source of the ultrasound signal. Some instruments may be flexible and/or may rotate as they navigate a twisting path to the treatment site, while others may change their angle of orientation to the ultrasound source. Multiple sensors may, in an embodiment, be positioned circumferentially around the needle or other medical tool to ensure at least one sensor will be oriented sufficiently to receive ultrasound signal from the probe. Multiple sensors may also be formed into an array in a suitable form factor for the device on which they are coupled. Furthermore, point sensors which may have up to an omni-receptive sensing feature (e.g., the ability to receive signals from all or substantially all direction) may be used instead of a series of optical sensor to still allow for the instrument to be tracked, regardless of its orientation to the probe.

The optical transducer 20 may be configured to detect acoustic signals generated from probe 100 in FIG. 1A. The optical transducer 20 may be configured to receive the acoustic signals through a photo-elastic effect and/or a physical deformation of the optical transducer 20. For example, in the presence of acoustic pulses or signals, light in the optical transducer 20 may undergo a spectral shift caused by changes in the refractive index and shape of the optical transducer 20. The optical transducer 20 may be configured to transmit a set of optical signals representative of the received acoustic signals to a processing system (e.g., the medical guidance processing system 200 in FIG. 1). In some examples, the optical transducer 20 may be coupled to one or more optical waveguides 22 (e.g., optical fibers, photonic integrated circuit waveguides, or other optical transmitting channel) to transmit the set of optical signals to the medical guidance processing system 200. The medical guidance processing system 200 may be configured to generate a real time location indicator for display at the display 300 based on the received optical signals. In some examples, the location indicator may be representative of a current position of the distal end of the instrument 10 and/or may be used to track the distal end of the instrument 10 (e.g., based on detected movement of the instrument 10). For example, the distal end of the instrument 10 may be visualized and tracked based on the location indicator. Accordingly, an instrument 10 may be reliably visualized and tracked during a medical procedure using at least a single optical transducer 20.

Optical transducers 20 configured to operate as polarization based (e.g., line type) sensors may operate as follows. Acoustic signals incident on exposed portions of an optical fiber (e.g., the optical sensors 20) may cause physical deformation and/or material property alteration of the polarization based sensors. Accordingly, an optical signal provided along the optical waveguide of the optical fiber may be altered by, influenced by, or otherwise indicative or representative of the acoustic signal incident on the optical fiber and therefore may be used to characterize the incident acoustic signal. In a polarization based sensor, the incident acoustic signal may cause stress in the optical fiber that results in one or more of birefringence and a rotation of the polarization of the light passing through the optical fiber. These changes in the polarization of the light carried by the optical waveguide of the fiber sensor may be detected and analyzed by the processing system 200.

When an optical transducer's location is known, the signal received by the optical transducer can be used together with signals received by elements in the ultrasound probe 100 for beamforming of ultrasound images, harmonics etc. Sensors such as the optical transducer 20 can be useful for harmonic imaging of surroundings because these transducers are very close to an imaging area of interest, and harmonic signals may be weak or unable to propagate very far. As commonly known, tissue, bone, implants and other structures in the area being insonified can cause scattering of acoustic signals and/or tissue harmonics. The optical transducer 20 can detect direct signals (e.g., from the ultrasound probe 100) and scattered signals and/or tissue harmonics resulting from the ultrasound probe 100 signals in the insonified area surrounding the optical sensor.

Additionally, visualizations of the tool or the sensor located on the tool can be displayed to assist the clinician, such as showing the path of the instrument 10 (e.g., the backward looking path of where the instrument 10 came from and/or the forward looking path of the predicted location of the instrument 10 based on a current trajectory) in the display 300 along with whether or not the instrument distal end is within the plane (e.g., within the imaging slice) of the beamforming signal.

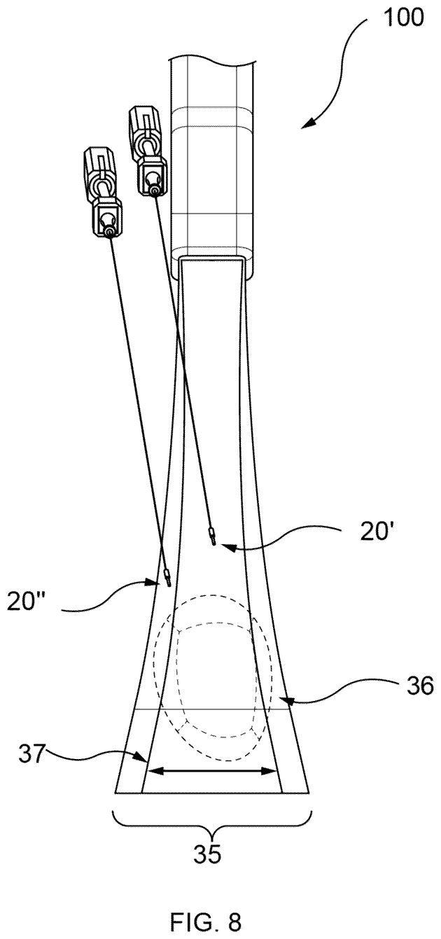

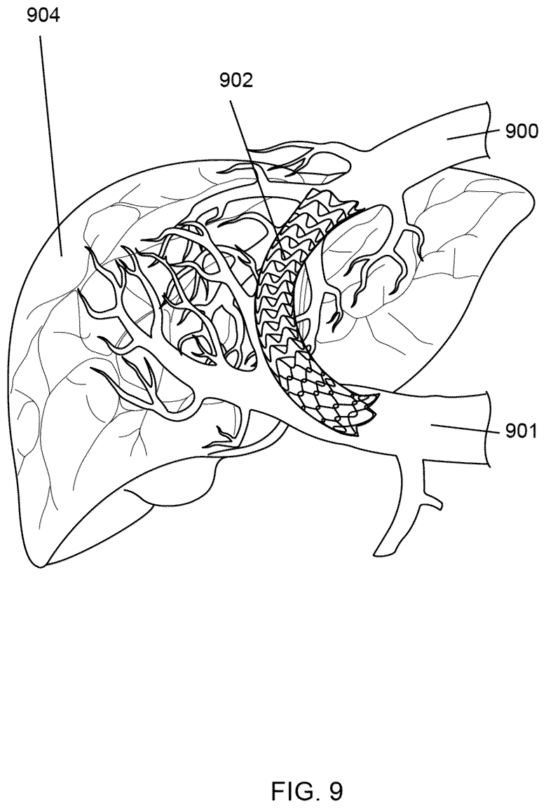

FIG. 8 is a schematic depicting an optical transducer 20′ within an imaging plane 35 and one optical transducer 20″ outside of the imaging plane 35, but within an insonified region 37. Because objects outside of the imaging plane 35 but still within the insonified region 37 may still be detected, it may be valuable to a clinician to be provided with information as to whether an optical transducer 20 is located in or out of the imaging plane 35. This allows for real time adjustment of the needle or instrument by the clinician to avoid anatomy or alter a path to the target area displayed in the ultrasound image. This may also be helpful if more than one tool is used in the procedure and they approach the procedure area from different directions. This may be referred to as ultrasound vectored needle guidance. The cartesian (e.g., xyz) position of the optical transducer 20 relative to the source (e.g., ultrasound probe 100) is known and thus the absolute position of multiple optical transducers 20 in relation to each other is also known. In FIG. 8, the out-of-plane transducer 20″ is depicted as out of plane in a lateral dimension. Out-of-plane transducers 20 in an elevation dimension may also be detected and labeled or indicated as such. In embodiments, different in-plane indicators may be used in the display 300 to indicate that an optical transducer is out of plane in a lateral or an elevation dimension. Examples of such procedures will be disclosed further with respect to FIGS. 9-23.

In some embodiments, an imaging system comprises the probe 100 and the optical transducer 20. A “delay-and-sum” beamforming method, or any other suitable beamforming method, may be applied to generate an ultrasound image of the surrounding medium (tissue) from the perspective of the optical transducer 20 and/or to generate information to enhance an ultrasound image generated based on acoustic signals received by the probe 100. In this imaging mode, ultrasound is transmitted from a probe/transducer array (which may include multiple transmits with different transmit patterns), and the medium/tissue scattering signal is received by the transponder sensor/sensors to form an ultrasound image. Signals from multiple optical transducers 20, or signals from the same optical transducer 20 but at different locations, can be coherently combined to form the ultrasound image. The locations of the optical transducers 20 are known or can be calculated at the time of signal acquisition. The optical transducers 20 can be a “point like” optical transducer 20a such as a fiber end Fabry Perot cavity sensor and/or a line type optical transducer 20b or 20c, such as a polarization sensitive fiber sensor. In the case of a “point like” transponder optical transducer 20a, a delay used to calculate the delay-and-sum beamforming corresponds to a straight-line distance from each pixel (or voxel in 3D imaging) to the transponder location (e.g., see FIG. 2A). In the case of a straight “line type” transponder sensor(s) 20b, a delay used to calculate the delay-and-sum beamforming corresponds to an orthogonal line distance from each pixel (or voxel in 3D imaging) to the transponder sensor(s) 20b line location (e.g., see FIG. 2B). If the “line type” transponder is curved, there may be multiple delay values for each pixel (or voxel) since there may be multiple orthogonal line paths from it to the transponder line optical transducer 20c (e.g., see FIG. 2C). In some configurations, the line type sensor(s) 20b and/or 20c is a simpler front-end design, optical detection is performed on the back end (e.g., using a polarization analyzer), and/or wavelength locking may not be required. By knowing a position of the fiber with respect to the ultrasound probe 100, and/or a timing sequency of emitters in the ultrasound probe 100, a location of tissue scattering can be calculated based on a propagation time of the acoustic signal (e.g., assuming the scattering signal is incident orthogonal to the optical fiber).

Various methods exist for determining the location of the optical transducer 20 based on the various signals and combinations of signals. In some examples, triangulation may be used to determine a position of one or more of the optical sensors. Ultrasound may be transmitted from the ultrasound probe 100, one or more external elements or array, or an in vivo array (e.g., an array for ICE, EBUS, EUS, IVUS). The transducers on the ultrasound probe 100 emit at least two signals with different wavefronts. The optical transducer 20 location is determined by the interception point of the different transmit wavefronts at respective received pulse timing. The pulse timing for the ultrasound transmission is determined by extracting and matching the known pulse shape from the transponder-received time sequence ultrasound signal. The pulse timing can be extracted when the pulse signal's signal-noise-ratio is higher than a certain number. A matched filter for known pulse shape or a Wiener filter can be used to enhance the pulse detection fidelity.

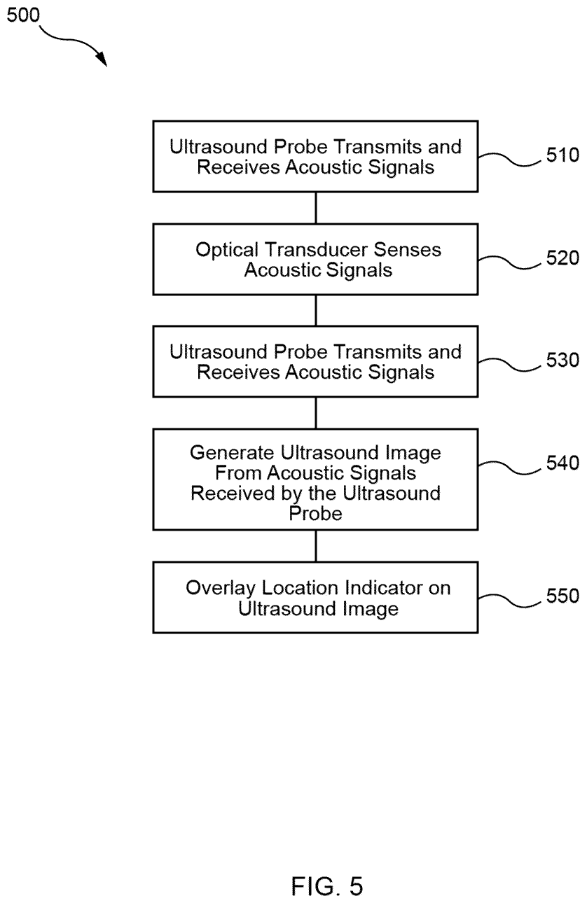

FIG. 4 is a schematic illustrating example positions of probe transducer elements 122 configured to emit acoustic pulses and an example position of an optical transducer 20 in a Cartesian coordinate system. The optical transducer 20 may be arranged on an object (e.g., instrument 10, not shown) to be tracked. The location of the optical transducer 20 may be determined using the Cartesian coordinate system.

In FIG. 4, the three probe transducer elements 122 are located at P1: (−a, 0, 0), P2: (a, 0, 0), P3: (0, b, 0), and the optical sensor is located at P: (x, y, z). The distances between the three transducer elements 122 and the optical transducer 20 may be calculated using the following equations:

$${r}_{1} = {( {( x + a )}^{2} + {y}^{2} + {z}^{2} )}^{1 / 2} \qquad eqn . ( 2 )$$

$${r}_{2} = {( {( x - a )}^{2} + {y}^{2} + {z}^{2} )}^{1 / 2} \qquad eqn . ( 2 )$$

$${r}_{3} = {( {x}^{2} + {( y - b )}^{2} + {z}^{2} )}^{1 / 2} \qquad eqn . ( 3 )$$

Solving Equation 1 and Equation 2 simultaneously results in:

$$x = ( {r}_{1}^{2} - {r}_{2}^{2} ) / 4 a \qquad eqn . ( 4 )$$

Equation 4 indicates that a≠0. That is, the distance between the first element and the second element cannot be zero. Solving Equation 1 and Equation 3 simultaneously results in:

$$y = ( {r}_{1}^{2} - {r}_{3}^{2} - {a}^{2} + {b}^{2} - 2 a x ) / 2 b \qquad eqn . ( 5 )$$

x in Equation 5 may be determined from Equation 4. Equation 5 indicates that b≠0. That is, the third element cannot be on the line determined by the first element and the second element. For example, the first, second, and third elements may form a triangle. Accordingly, the third element is offset in a first dimension (e.g., elevation dimension). Therefore, from Equation 1:

$$z = {( {r}_{1}^{2} - {( x + a )}^{2} - {y}^{2} )}^{1 / 2} \qquad eqn . ( 6 )$$

where x and y are determined from Equation 4 and Equation 5.

If the acoustic velocity is c and the time required for an acoustic beamforming pulse to travel from the first element to the optical sensor is t₁, then:

$${r}_{1} = c {t}_{1} \qquad eqn . ( 7 )$$

r₂ and r₃ may be determined in a similar manner as r₁. Therefore, the location of the optical transducer 20 may be determined based on the time required for an acoustic pulse to travel from an element 122 to the optical transducer 20.

Although the location of the optical transducer 20 may be determined by detecting acoustic signals (e.g., echoes) corresponding to acoustic pulses from three probe transducer elements 122, in some examples, more than three elements 122 may be used to determine the location of the optical transducer 20. The elements 122 may be positioned in any suitable manner. However, in such a triangulation technique, to enable tracking of optical transducer 20 in a 3D space, elements 122 and the optical transducer 20 cannot be in the same plane. For example, a first and second element may be arranged along a lateral dimension and a third element may be arranged along an elevation dimension transverse to the lateral dimension where the third element does not intersect the lateral dimension (e.g., so as to be arranged as vertices of a triangle). Accordingly, the third element in this example is not aligned with respect to the lateral dimension of the first and second elements. The first and second elements are offset with respect to each other but are aligned in the lateral dimension. In some examples, using more than three elements 122 may improve the accuracy of the determined location of the optical transducer 20. In some examples, more than one optical transducer 20 may be used to detect acoustic signals. The position of each optical transducer 20 may be determined similar to as described above. If probe transducer elements 122 and the optical transducer 20 are in the same plane, 2D tracking information within that plane can still be obtained. In this case, at least two transducer elements 122 are used.

In another example, the location of the optical transducer 20 is determined by coherent image forming. Features are most easily identified in ultrasound images when they differ in image brightness. The intensity of the image in ultrasound imaging system is a function of the amplitude of the beamformed received signal, i.e. the amplitude after coherent addition of the delayed received signal from each transducer element.

In one example, multiple ultrasound firing is transmitted by the external elements or array on the probe 100 and from different locations and/or directions, and with different wavefront (similar to ultrasound imaging transmit sequences). For each pixel in the imaging plane, the pixel values are calculated from the transponder-received signal of the multiple transmissions, with the assumption that the optical transducer 20 is at the location of that pixel. The obtained image (transponder signal image) adds signals coherently only at the true transponder location where the received signal aligns, and ultrasound interference is constructive. The transponder signal image allows optical transducer 20 position determination because only the transponder location will light up in the image (with the ultrasound physics limiting the transponder image spot size). A single point transponder location can be extracted from the bright transponder spot in the transponder signal image by different methods (e.g., maximal pixel value, median filter, center of brightness weight, etc.). The advantage of using the coherent transponder tracking image is that the received transponder signal from different transmit is first added coherently, and then the pulse timing is determined on the coherently summed signal where the signal-to-noise ratio (SNR) is much higher than a single received time sequence signal. When the external elements/array operate with an imaging firing sequence, an ultrasound image can be generated at the same time of transponder tracking. Thus, there is no dedicated transponder tracking firing sequence. This coherent beamforming transponder imaging method can also be used for 3D tracking of the transponder. In the 3D case, the probe 100 will have (e.g., at least) three probe transducer elements 122, and (e.g., at least) one probe transducer element 122 is outside the plane defined by the optical transducer 20 and another two probe transducer elements 122 of the probe 100 are in plane as shown in FIG. 4.

In one example, the acoustic sensing signal received by the optical transducer 20 from different transducer elements 122 of the probe 100 are summed at the processing system 200 so that a net signal representing the ultrasound signal emitted from each transducer element 122 of the probe 100 is obtained. The sum of the amplitude of the summed signal represents the intensity of the signal received and thus corresponds to the distance along the beam associated with the signal at the angle from the optical transducer 20 to the probe transducer element 122. Summing of the individual signals is accomplished by providing separate time delay (and/or phase) and gain to the signal from each transducer element 122 in the probe 100. The output signal from the optical transducer 20 corresponding to each beam forming channel is then coherently added, i.e., each channel is summed, to form a respective pixel intensity value for each beam. The pixel intensity values can be logarithmically compressed, scan converted, and then displayed as an image of the distal end of the instrument 10 where the optical transducer 20 is located or the entire needle when multiple optical transducers 20 are utilized.

In some examples, there can be multiple transponders, such as the optical transducers 20 coupled to instrument 10 in FIG. 3B, each operating and receiving signals independently. The optical transducers 20 can share or receive the same external elements or array firing sequence signals from probe 100 for tracking each of their respective locations. Coded excitation may be used to increase the signal-to-noise ratio (SNR). Such coded excitation may be used in conjunction with a long or multi-pulse, chirp-signal technique for the ultrasound firing sequences. The received transponder sensor signals can be applied to a matched filter/Weiner filter for pulse compression to achieve a much higher SNR for the pulse timing determination and/or a much better axial resolution in the beamformed transponder signal image. The resulting higher SNR can increase transponder tracking accuracy.

One or more electrical signals can be generated as sensor data based on one or more detected optical responses to light propagation within one or more optical transducers 20 in response to one or more acoustic signals incident on the one or more optical transducers 20. The sensor data can be used to enhance an ultrasound image. For example, the probe 100 is used to generate an ultrasound image (e.g., a first image); sensor data is used to generate a sensor image (e.g., a second image; based on known time and location generation of acoustic pulses from the probe 100 and/or a known location of the optical transducer 20 with respect to the probe 100); and the sensor image is combined with the ultrasound image (e.g., by image fusion using processor 110 in FIG. 1B) to enhance the ultrasound image to generate an enhanced image (e.g., a third image; to increase resolution of an area in the ultrasound image near the optical transducer 20). In some embodiments, sensor data is sent to the processor 110 in FIG. 1B without generating a sensor image (e.g., the processor 110 generates the enhanced image based on the sensor data and data from the ultrasound probe 100 so that one image, the third image, is generated and the first image and/or the second image is not generated separately from the third image). In some cases, the first image (the ultrasound image) and third image (the enhanced image) are generated without the second image (the sensor image). In some cases, the second image (the sensor image) is generated without generating the third image (the enhanced image) or the first image (the ultrasound image).

In embodiments, a device path can be ascertained by the information obtained by the optical transducer 20. When an optical transducer 20, or multiple optical transducers 20s, are integrated on a device (e.g., a needle, catheter, etc.), the location history of the transponder sensor/sensors may be used to determine the path the device has taken. The history path can be used to provide valuable medical information. In some applications, it can be used to predict the device movement. For example, when a needle has travelled a certain distance, using its location history, a projected needle path can be predicted and/or overlayed on the ultrasound image. In doing so, one can assume, in some embodiments, the needle is taking a straight path, or a curved path that can be defined by the history locations. Additionally this information may be used to project the current expected path given the current path. The history path can also be used to indicate the physiological structure the device has gone through. For example, a catheter device travelling through a blood vessel can map the shape of the vessel from the history path of the device transponder sensor. The history path of a device can also serve as records of medical operation and/or to evaluate operation performance and safety. For example, the history of the two transponders on the two sides of a forceps can be used to determine how many times they have closed/opened. Further, for procedures involving multiple insertions, history paths may be used to differentiate previous paths and/or to allow an operator to successfully choose a new path. Additionally, history paths may assist during the use of multiple tools. History paths and projected paths may assist in concurrent navigation of multiple tools.

Information captured by one or more optical transducers 20 can be used to ascertain the shape and/or orientation of the device. When an optical transducer 20 or multiple optical transducers 20 are integrated on a device (e.g., a needle, catheter, etc.), the locations of the optical transducers 20 can be used to ascertain the shape and/or orientation of the device. For example, when multiple optical transducers 20 are integrated along a catheter, their locations can be used to ascertain the shape of the catheter (e.g., point-by-point curve). The shape of the catheter can then be used to ascertain the shape of the physiological structure it is in, for example a blood vessel or a lung bronchus. In another example, the locations of two optical transducers 20 on a needle can be used to ascertain the orientation and position of the needle (e.g., assuming the needle is a straight line). The locations of three optical transducers 20 can be used to ascertain the orientation and position of a surface of a medical device (three points form a surface), or the medical device itself if it is a rigid body. When a polarization line sensor is used, multiple transmits can be programmed to emit from a probe to “scan” the line sensor. Since the line sensor is sensitive to ultrasound that laterally arrives at the sensor, the “scan” will generate signals at the sensor when the transmitted ultrasound is lateral to part of the line, therefore locating the section of the line that is lateral to a specific transmit pattern. When the positions and orientations of multiple sections of a line are ascertained from multiple transmit patterns, the shape and position of the line can be ascertained/estimated from the sectional information. The shape and position of the line sensor can therefore be used to indicate the shape and position of a medical device that integrates the line sensor.

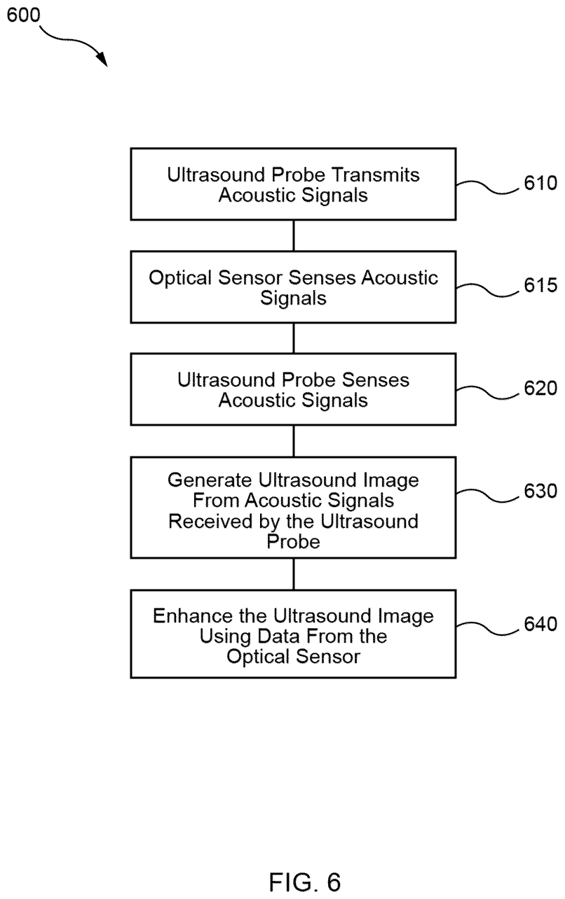

Referring now to FIG. 5, FIG. 5 shows an example method 500 for optical transducer tracking and ultrasound image enhancement. This example method 500 will be described with respect to the system shown in FIGS. 1A, 1B and 2; however, another suitable system according to this disclosure may be employed.

In an operation 510, an ultrasound probe (e.g., an external or in-vivo probe) transmits and receives acoustic signals. For example, the ultrasound probe 100 shown in FIG. 1A transmits acoustic pulses from an array of transducers into the medium 5, which represents the anatomy of a patient. The ultrasound probe 100 may transmit these pulses using a variety of known methods or as described above. The ultrasound probe 100 receives the acoustic signals (e.g., probe 100 receives acoustic signals reflected or scattered from objects and/or features, such as tissue, in the medium 5). For example, echoes might be reflected off of a tumor present in the medium. The ultrasound probe 100 converts the ultrasound pulses to signals that are then transmitted to the processing system 200.

At operation 520,one or more optical transducer senses acoustic signals. For example, the optical transducer 20 coupled to the instrument 10 in FIG. 1A also receives ultrasound pulses (e.g., acoustic beacon signals) that were emitted by the probe 100. The optical transducer 20 converts the ultrasound pulses to signals that are then transmitted to the processing system 200. In embodiments, as discussed above, multiple instruments 10, each having one or more optical transducers 20, may be included in the operation 520.

At operation 530, the medical guidance processing system 200 determines the location of the optical transducer based, at least in part, on the signals received from optical transducer 20. For example, the medical guidance processing system 200 may utilize triangulation and/or beamformed methods to determine the position of the optical transducer based on a plurality of signals received from the optical transducer 20.

At operation 540, the medical guidance processing system 200 generates an ultrasound image. For example, the ultrasound image is generated from acoustic signals received by the probe 100. The ultrasound image may be transmitted to and displayed on the display 300. Alternatively, in some embodiments, the medical guidance system 200 may obtain an anatomical image from another source, e.g., a CT or fluoroscopy image.

At operation 550, the processing system 200 overlays the location of the optical transducer over the ultrasound image (or an alternative anatomical image). For example, a location indicator, a graphic, such as cross hairs (e.g., “+”) or a circle, is overlayed on the ultrasound image to correspond to a location of the needle distal end 14 in the ultrasound image. In-plane and out-of-plane indicators, location history, predicted location, trajectory, orientation information, and other location tracking information may also be displayed. As discussed above, location indicators may be provided for multiple instruments 10 corresponding to multiple optical transducers 20. In embodiments, the location indicators (combined with in-plane indicators) may be indicative of x-y-z locations within the anatomy corresponding to the ultrasound image. In embodiments, the x-y-z coordinates may be displayed in conjunction with the location indicators. Thus, when viewed by a user, such as an ultrasound technician, medical personal, or patient, the location of the one or more optical transducers is shown on the same display as the ultrasound image, indicating where in the medium 5, the optical transducer, optical transducer 20 on instrument 10, is located. The image may also display the path and/or projected path.

Referring now to FIG. 6, FIG. 6 shows an example method 600 for ultrasound image enhancement with a point sensor (e.g., using a fiber end optical sensor) or a line sensor (e.g., using polarization in an optical fiber or multiple point sensors). This example method 600 will be described with respect to the systems shown in FIGS. 1A and 1B, however, another suitable system according to this disclosure may be employed. As discussed above, he optical transducer 20 can detect scattered signals and tissue harmonics.

At operation 610, an ultrasound probe (e.g., an external or in-vivo probe) transmits acoustics pulses. For example, the ultrasound probe 100 shown in FIG. 1A transmits acoustic pulses from an array of transducers into the medium 5, which represents the anatomy of a patient. The ultrasound probe 100 may transmit these pulses using a variety of known methods and/or as described above. At operation 615, the optical transducer (e.g., point sensor or line sensor) senses the direct acoustic signals (e.g., from a probe 100), acoustic signals reflected and/or scattered from objects and/or features such as tissue in the medium 5, and/or tissue harmonics. For example, echoes might be reflected off a tumor present in the medium 5 in FIG. 1. The point type sensor will receive scattering from any direction or axially as shown in FIG. 2A while the line sensor will receive scattering from orthogonal or transverse directions as shown in FIGS. 2B and 2C.

When an optical transducer's location is known, the optical transducer signal can be used together with signals received by elements in the ultrasound probe (e.g., ultrasound probe 100 in FIG. 1) for beamforming of ultrasound images, harmonics etc. Optical transducer sensors can be useful for harmonic imaging of surroundings because optical transducers are very close to an imaging area of interest, and harmonic signals may be weak or unable to propagate very far. Tissue scattering can cause scattering of acoustic signals and/or tissue harmonics. The fiber sensor can detect direct signals (e.g., from a probe), scattered signals, and/or tissue harmonics.

At operation 620, the ultrasound probe senses acoustic signals. Signals (e.g., electrical and/or optical from the transducer and/or the probe), that correspond to sensed acoustic signals, are transmitted to a processing system (e.g., medical guidance system 200 in FIG. 1A). In some embodiments, a point-like sensor (e.g., optical transducer 20 in FIG. 1A or 20a in FIG. 2A) is also used to calculate and overlay a position of a device (e.g., as described in conjunction with FIG. 4).

At operation 630, the processing system 200 generates an ultrasound image. For example, the ultrasound image is generated from acoustic signals received by the ultrasound probe 100 in Figure A1.

At operation 640, the processing system 200 enhances the ultrasound image to generate an enhanced ultrasound image. The processing system 200 uses data from the fiber optical transducer 20 to enhance the ultrasound image. This data includes the direct signals and scattered signals. The enhanced ultrasound image may be transmitted to and displayed on the display 300. The data from the fiber sensor may also be used to create a separate image of the insonified region surrounding the sensor that is then transmitted to and displayed on display 300.

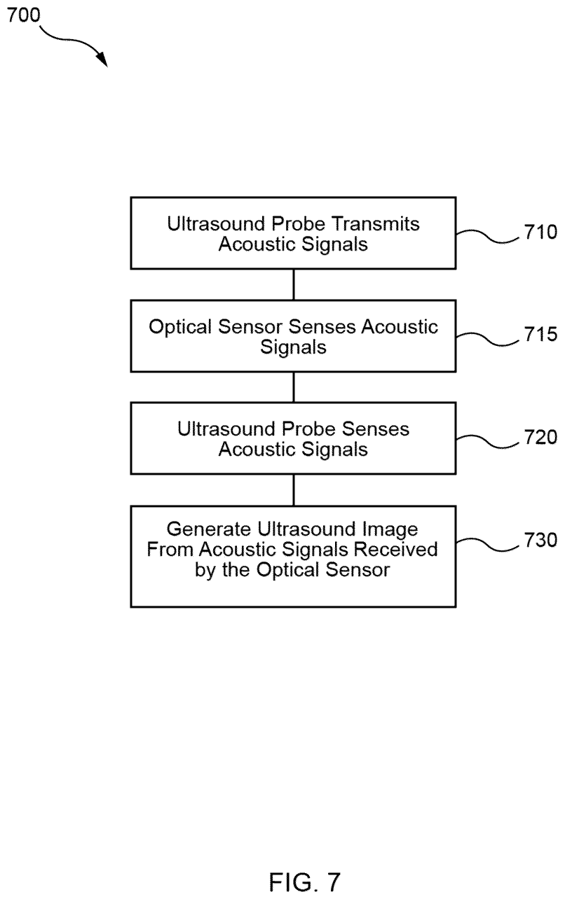

Referring now to FIG. 7, FIG. 7 shows an example method 700 for generating an ultrasound image of the insonified region surrounding the optical transducer that may be a point sensor (e.g., using a fiber end optical sensor) or a line sensor (e.g., using polarization in an optical fiber or multiple point sensors).

At operation 710, an ultrasound probe (e.g., an external or in-vivo probe) transmits acoustics pulses. For example, the ultrasound probe 100 shown in FIG. 1A transmits acoustic pulses from an array of transducers into the medium 5, which represents the anatomy of a patient. The probe 100 may transmit these pulses using a variety of known methods and/or as described above. At operation 715, the ultrasound transducer (e.g., point sensor or line sensor) senses the direct acoustic signals (e.g., from a probe 100), acoustic signals reflected and/or scattered from objects and/or features such as tissue in the medium 5, and/or tissue harmonics. For example, echoes may be reflected off a tumor present in the medium 5 in FIG. 1. The point type sensor will receive scattering from any direction or axially as shown in FIG. 2A while the line sensor will receive scattering from orthogonal or transverse directions as shown in FIGS. 2B and 2C.

At operation 720, the ultrasound probe, e.g., the external probe, senses acoustic signals. Signals (e.g., optical signals that are converted to electrical signals), that correspond to sensed acoustic signals, are transmitted to a processing system (e.g., medical guidance processing system 200 in FIG. 1). These signals may be used to assist in generating an ultrasound image from the signals received by the optical transducer/sensor. In embodiments, operation 720 may be optional and the method may move directly from operation 715 to operation 730.

At operation 730, the medical guidance processing system 200 generates an ultrasound image. For example, the ultrasound image is generated from acoustic signals received by the optical transducer/sensor. The ultrasound image may be transmitted to and displayed on the display 300. In embodiments, the ultrasound image may further be generated with additional information obtained from the acoustic based external ultrasound transducer.

The foregoing describes the advantages of on-tool ultrasound based optical sensors. Such sensors can provide accurate real-time tracking, orientation, and location of tools on which the sensors are located. Such sensors may also be used to provide enhanced ultrasound imagery and tool-perspective ultrasound imagery. Each of these advantages may be particularly helpful during various medical procedures. These advantages may improve the speed, accuracy, safety, and efficacy of various medical procedures. Accurate real-time tracking, orientation, and location of tools may be particularly useful during any type of procedure that makes it difficult to image or track a tool and/or anatomy, as well as their relationship to one another. Further, any procedure which may require multiple tools working together may be enhanced by the on-tool ultrasound based optical sensors described herein, e.g., by using multiple ultrasound transducers to separately guide the multiple tools to the appropriate location within the anatomy and singular or multiple ultrasound transducers to image the interaction between the multiple tools. Examples of procedures that may benefit from the on-tool ultrasound based optical sensors described herein are provided below. The following list is provided by way of example only and is not limiting. A person of ordinary skill in the art will understand that the on-tool ultrasound based optical sensors and methods of use discussed below may equally apply to different or varied medical procedures.

FIGS. 9-13B illustrate the use of embodiments, methods, devices and systems described herein to improve a transjugular intrahepatic portosystemic shunt (TIPS) procedure. In a TIPS procedure, an interventionalist builds a new blood vessel 902 between the portal vein 901 and the hepatic vein 900 to bypass a sick or damaged liver 904, as shown in FIG. 9. A portion of this procedure requires that a needle be pierced through the liver from the hepatic vein 900 to the portal vein 901 to create a connection around which the new vessel can be built. The procedure is typically carried out by remote tools guided by the interventionalist and inserted through an incision in the neck into the jugular vein. Current imaging techniques employed in this procedure, such as CO2 portography, traditional ultrasound, intracardiac echo (ICE) ultrasound, gunsight/fiducial imaging, X-ray imaging, and others all have significant drawbacks. For example, some techniques lack live visualization, some techniques fail to image all relevant portions of the anatomy (e.g., the portal vein 901), some techniques use excessive amounts of X-Ray radiation, resulting in significant exposure of both the patient and the interventionalist, etc. Further, some procedural locations may lack the equipment (e.g., x-ray imaging equipment) to carry out this procedure under x-ray imaging. This lack of imaging typically leads to the interventionalist repeatedly attempting to pierce the needle from the hepatic vein 900 to the portal vein 901 through the liver 904 until the connection is made. Each additional attempt bears the risk of piercing the main portion of the portal vein 901, occluding the hepatic artery, and/or causing other trauma to the patient. Such mishaps can result in patient death. The ultrasound based tools discussed herein provide operators with the ability to carry out this procedure with only ultrasound based tools without the need to invest in more expensive equipment.

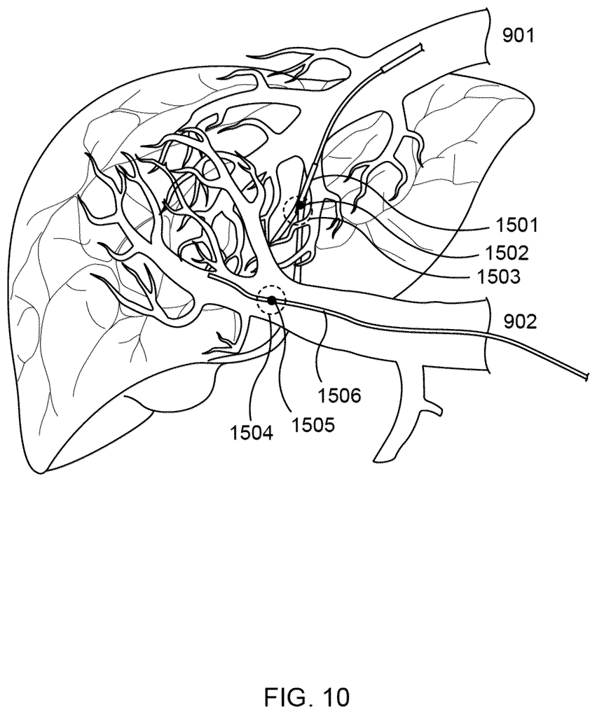

Systems and methods discussed herein may be applied to this procedure to significantly increase its safety and success, as shown in FIGS. 10 and 11A and 11B. FIG. 10 illustrates aspects of a TIPS procedure performed with surgical tools including optical based ultrasound sensors as described herein. FIG. 11A and FIG. 11B illustrate examples of ultrasound location tracking and image forming. A surgical tool 1501 including a distal needle may include an optical based ultrasound transducer 1502. The optical based ultrasound transducer 1502 (e.g., an instance of the optical transducer 20) may include any optical based ultrasound sensors, transducers, and/or arrays discussed herein, including, e.g., fiber end sensors as well as mixed array transducers. A surgical tool 1506 may also include an optical based ultrasound transducer 1505. The surgical tool 1506 may be, for example, a guidewire or similar tool. The optical based ultrasound transducer 1502 and the optical based ultrasound transducer 1505 may be disposed at any suitable location on their respective surgical tools 1501/1506 to facilitate the tracking and monitoring as discussed herein.

During the TIPS procedure, the systems and methods described herein may be implemented to improve the ease, speed, safety, and accuracy of the procedure. As shown in FIG. 10, during the procedure, the surgical tool 1501 may be tracked through the hepatic vein to the procedure location. The surgical tool 1506 may be tracked through the portal vein to the procedure location. Through the use of ultrasound beacon signals as described herein, a location 1503 of the sensor 1502 may be visualized and tracked and a location 1504 of the sensor 1505 may be visualized and tracked. FIG. 11A illustrates a location indicator 1510 representative of the location 1503 including an in-plane indicator 1511. The location indicator 1510 (e.g., the circle) shown in FIG. 11A includes an in-plane indicator 1511 (e.g., the hash marks) that indicate how close the located needle is to the image plane. In this embodiment, as the located needle approaches the imaging plane, the number of hash marks is reduced until there is only the circular location marker when the located needle is aligned with the imaging plane. In embodiments, the location indicator 1510 may also change color when plane alignment is achieved. Accordingly, during a TIPS procedure, the surgical tool 1506 may be guided to a location in the portal vein, e.g., location 1504, to provide a reference location which the surgical tool 1501 is to be steered/navigated to. The interventionalist may then track the changing location 1503 of the surgical tool 1501 to guide the needle at the distal end of the surgical tool 1501 to the location 1504. In embodiments, the changing location may be represented both by the location indicator 1510 and by display of the x-y-z coordinates. This x-y-z localization information may be displayed in real time or substantially real time as to where the distal end of the surgical tool 1501 is required to move to make the puncture between the portal vein 901 and the hepatic vein 900. The interventionalist may further be aided by predicted trajectory, historical path, and orientation indictors, as discussed herein. The interventionalist may be further aided through use of the ultrasound beamforming signals to generate an ultrasound image from the beamforming signals received by either or both of the sensor 1502 and the sensor 1505, e.g., as shown in FIG. 11B. The image shown in FIG. 11B is from the forward looking perspective of the distal needle at the end of surgical tool 1501. Thus, the interventionalist may not only track and monitor the location of each surgical tool 1501/1506, they may also receive ultrasound images captured by the surgical tools to assist in guiding the tools through the patient's anatomy. In this way, the interventionalist may more easily, quickly, and safely create the necessary connection between the hepatic vein and the portal vein before completing the surgical procedure by building the new blood vessel shunt.

Another type of procedure that may benefit from the systems and methods disclosed herein may be any type of deep tissue biopsy procedure. For example, traditional biopsy procedures in the abdomen of patients may become complicated when there is a significant amount of tissue surrounding the biopsy target. Such may be the case, for example, when a patient is overweight or obese. Excess tissue in the abdomen may create difficulties for the interventionalist when using traditional ultrasound methods. Due to the excess tissue, traditional ultrasound methods may not provide accurate enough imaging to guide a surgical tool to deeply located areas, such as the liver, kidneys, gall bladder, etc. Because of the deep location of these biopsy targets, a traditional ultrasound signal, provided by an external probe, must travel through a significant amount of tissue to reach the target and reflect back. Using techniques described herein, wherein optical based ultrasound transducers are located directly on a tool to be guided, such procedures can be improved. For example, with an on-tool transducer, an ultrasound signal generated by an external probe/transducer is only required to travel half the distance to be received by the on-tool transducer as compared to a traditional external probe.

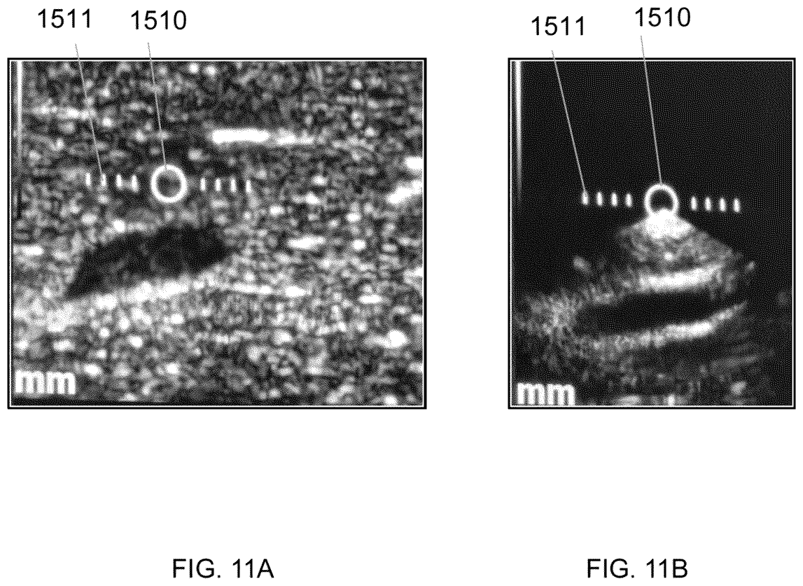

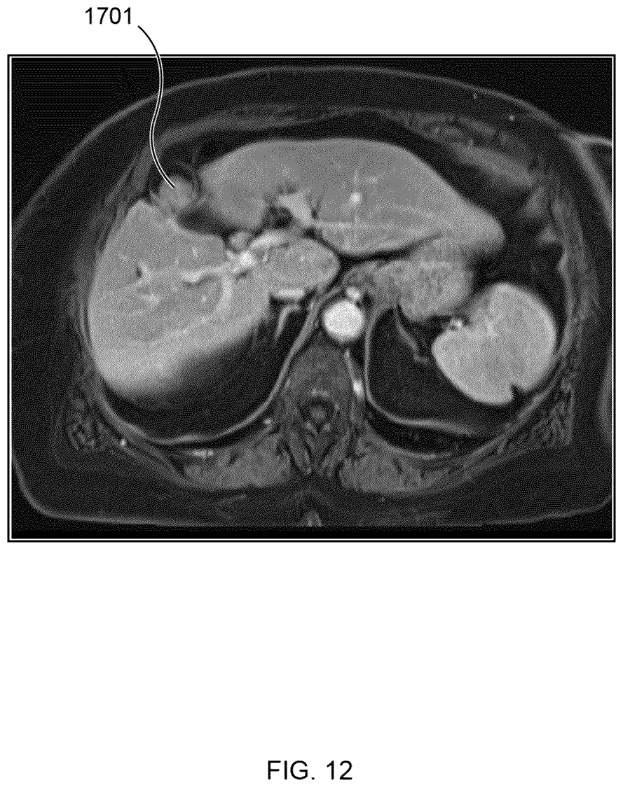

FIG. 12 illustrates an axial view of a patient abdomen. Located within the abdomen is a gall bladder 1701, which may be an example of a deep tissue biopsy target. FIGS. 13A and 13B show examples of tracking a surgical tool with an on-tool optical based ultrasound transducer. In FIG. 13A, the location indicator 1301 includes an in-plane indicator 1302 having hash marks that indicate that the surgical tool is out of plane with the ultrasound image. In FIG. 13B, the location indicator 1301 does not include the hash marks, indicating that the surgical tool is in plane with the ultrasound image.

In additional embodiments, methods and systems discussed herein may be employed to facilitate and improve ablation procedures. Ablation procedures, such as RF, microwave, or cryo ablation procedures, may benefit from the tools described herein in several ways. First, ablation procedures may benefit from the increased location accuracy provided by the on-tool optical based ultrasound transducers described herein to ensure that ablation tools are appropriately located within an ablation target. In addition to the increased location accuracy, real-time or near-real time tracking or location monitoring may be beneficial to ensure that the ablation tool is not displaced from the target location during a procedure. Further, optical based ultrasound transducers may be provided at the end of optical fibers that are non-conductive of RF and microwave energy. Thus, the RF and microwave signals generated for ablation procedures does not interfere with the optical signals used in the optical based ultrasound transducers. Some location technologies that include traditional conductive wires may suffer from interference from the ablation energy. Additionally, as described herein, techniques may be used to account for temperature variations of the optical based ultrasound transducers. In an ablation environment, both the target tissue and the ablation tool undergo significant heating. Such heating may distort results from some types of sensors used for location purposes during an ablation procedure. Additionally, any effect on the optical sensor by the cryotherapy may be accounted for when processing the sensor signals. In a further advantage, the on-tool optical based ultrasound transducers may be used to measure temperature, e.g., as described in patent application Ser. No. 18/685,985 , titled “MULTI-DIMENSIONAL SIGNAL DETECTION WITH OPTICAL SENSORS.” Such temperature measurement may be used to determine the extent of the ablation effect, e.g., based on tissue temperature. Such temperature measurement may be used to track and identify the boundaries of tissue isotherms created during an ablation procedure. Temperature measurements may further be used to detect the extent of tissue damage after ablation completion. Rapid tissue cooling may be a sign of returning blood flow to an area, which may indicate that an ablation procedure has left too much tissue intact. Temperature measurement via optical sensors may have advantages over more traditional temperature transducers for several reasons. First, by using the optical sensors for temperature measurement, it may be unnecessary to provide additional temperature sensors and wiring which may lead to a bulkier ablation tool. Next, as discussed above, optical based sensors are robust against interference from the RF and microwave energy generated during an ablation procedure. In embodiments, such temperature measurement optical transducers may be located on the ablation tool itself and/or may be provided in separate probes. In some cases, the heat of an ablation procedure may damage optical transducers. Accordingly, separate probes including optical transducers may be advantageous. All of the advantages discussed above may adhere to temperature measurement optical transducers positioned on separate probes. In addition, such separate probes may permit alternate positioning of the transducers. A temperature measurement probe located on the ablation tool itself may not be enough to determine the extent of ablation. Separate probes may be positionable around the edges of a tumor or other site to be ablated. The location capabilities of the optical transducers may ensure accurate location while the temperature measurement capabilities may be used to detect when the ablation related temperature increase has extended past the bounds of the tumor. In some embodiments, each of such separate probes may include multiple optical transducers, increasing the positioning flexibility.

Any ablation procedure may benefit from the on-tool optical based ultrasound transducers described herein. One example of such includes thyroid ablation. Thyroid ablation may particularly benefit from the tools and techniques described herein due to the unique challenges associated with such ablations. First, because of the non-circular shapes of thyroid nodule ablation targets, a “moving shot technique” is often used. The moving shot technique is used to move the ablation tool during the ablation procedure to create multiple ablation units, thereby permitting the interventionalist to shape the ablated area. Enhanced location accuracy and tracking permitted by on-tool optical based ultrasound transducers can facilitate and improve this technique by increasing the ability to visualize and track the ablation tool. Tracking history information may be used to accurately mark areas that have previously been ablated. Further, the thyroid is a relatively small portion of tissue with many adjacent structures that an interventionalist tries not to damage - the vagus nerve, the esophagus, the common carotid artery, the inferior jugular vein, the anterior jugular vein, the trachea, the “danger triangle” including the recurrent laryngeal nerve, and others. Careful and accurate location of the ablation tool, facilitated by an on-tool optical based ultrasound transducer, may aid the interventionalist in avoiding these areas.

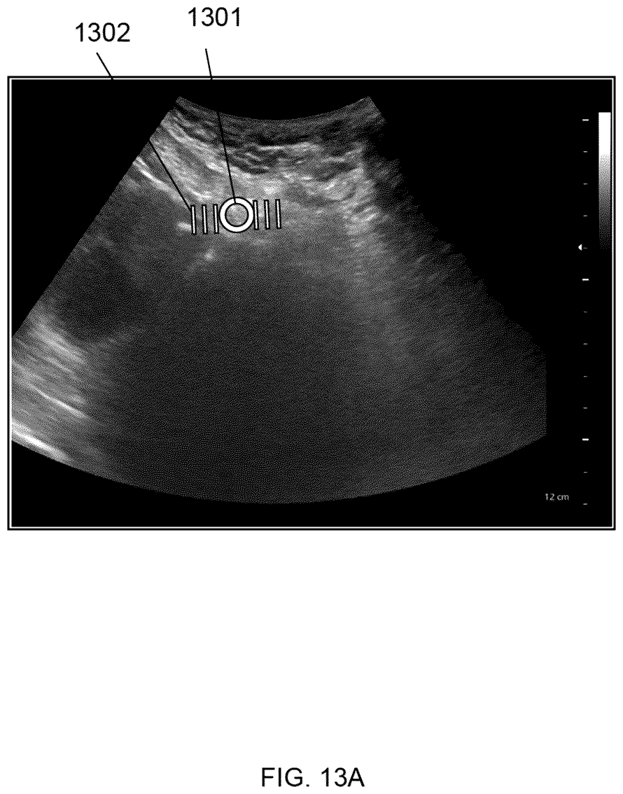

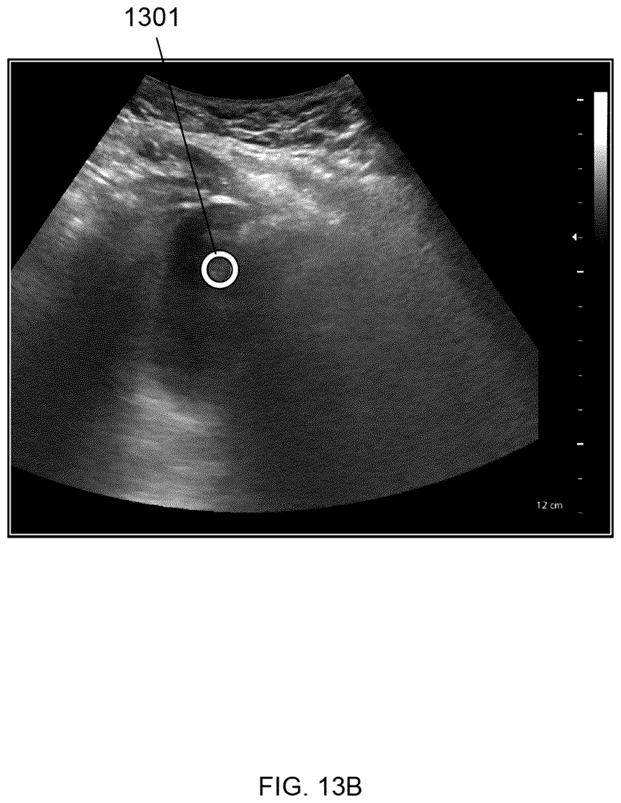

Aspects of a thyroid ablation procedure are illustrated in FIGS. 14A and 14B. FIG. 14A illustrates a moving shot technique while FIG. 14B shows the thyroid amid adjacent structures that can be damaged during an ablation procedure. FIG. 14A illustrates an ablation tool 1901 and three ablation locations 1902, 1903, and 1904. The ablation tool 1901 includes an on-tool optical based ultrasound transducer to facilitate accurate location tracking. The ablation tool 1901 may be any suitable ablation tool, including RF, microwave thermal, etc. The ablation tool 1901 is shown in triplicate in FIG. 14A, extending to each of ablation locations 1902, 1903, and 1904. In embodiments, previous ablation locations may be marked according to a location history of the ablation tool 1901. Previous paths of the ablation tool 1901 may be displayed to assist the interventionalist in finding a path to a new ablation location. During a procedure, the ablation tool 1901 may be repositioned several times to ensure that the pattern of ablated tissue adequately covers the thyroid nodule. The interventionalist may use the accurate location tracking provided by the on-tool optical based ultrasound transducer to accurately position the ablation tool 1901 within the thyroid while avoiding the adjacent anatomical structures 1905 to be avoided. These adjacent anatomical structures 1905, which the interventionalist seeks to avoid, are shown in greater detail in FIG. 14B. In addition, internal imaging from the optical transducer located on the ablation tool, both the point-of-view imaging and the enhanced imaging discussed herein, may be employed to assist in confirmation of the size and extent of an ablation lesion.

Another procedure that may benefit from the use of on-tool optical based ultrasound transducer techniques described herein is a nephrostomy procedure. During a typical nephrostomy procedure, a needle is inserted percutaneously through the renal parenchyma into the patient's calyx under ultrasound guidance. A guidewire is inserted through the needle and guided through the collecting system, often into the renal pelvis or ureter. This portion of the procedure may be performed under ultrasound or x-ray fluoroscopy. After placement of the guidewire, a catheter is inserted into the collecting system and secured in place for urine drainage.

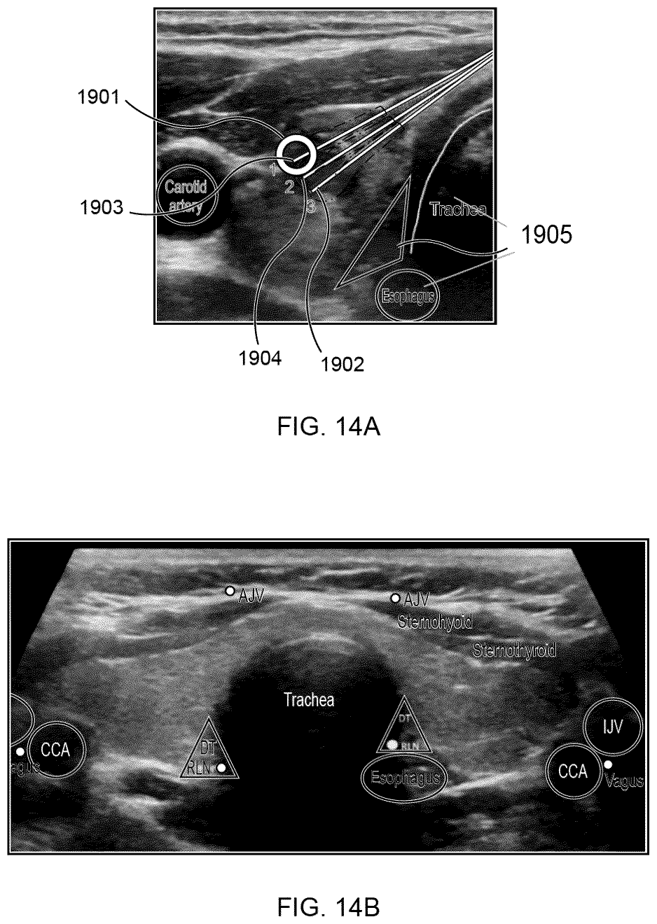

FIGS. 15A-15C are ultrasound images of a kidney. As can be seen, the interior anatomy of the kidney can vary significantly. In some instances, the interior anatomy of the kidney can make placement of the needle, guidewire, and/or catheter difficult. FIG. 15A shows a kidney with a well dilated collecting system that may facilitate an easier procedure. FIG. 15B shows a kidney for which the procedure may be more difficult due to the more closed nature of the collecting system and FIG. 15C shows a kidney for which the procedure may be very difficult. The locational accuracy provided by on-tool optical based ultrasound transducer techniques discussed herein may provide the interventionalist with the ability to perform a nephrostomy procedure in even the most difficult anatomical cases.

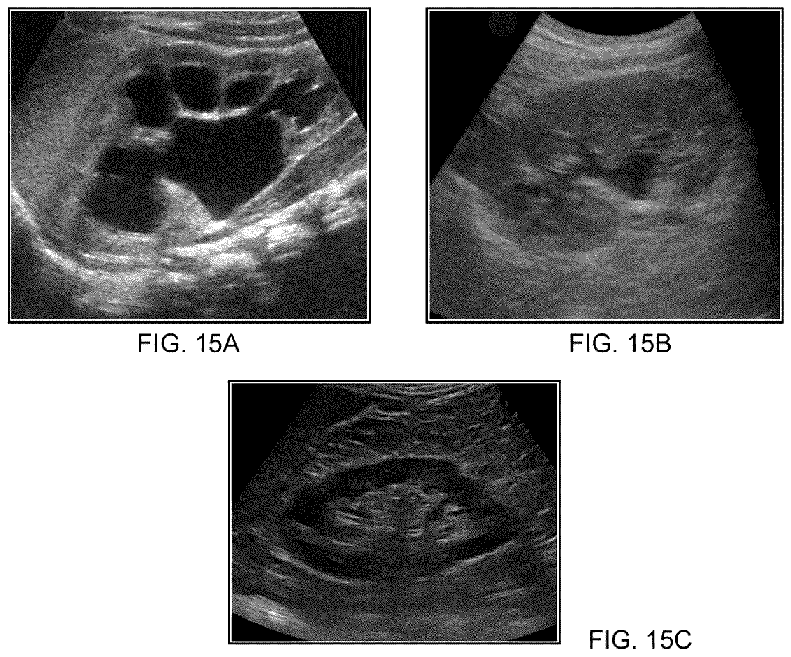

FIG. 16 illustrates surgical tools that may be used in a nephrostomy procedure. Surgical tool 2101 is a needle that may be used to perform the initial portion of the procedure, i.e., insertion into the kidney. The surgical tool 2101 includes an on-tool optical based ultrasound transducer 2103 configured to receive beacon signals and beamforming signals, as discussed herein. Received beacon signals and beamforming signals may be processed and used to assist in the navigation of the surgical tool 2101 to the appropriate location for a nephrostomy procedure. Surgical tool 2102 is a catheter that may be inserted into the collection system of the kidney to drain urine. The surgical tool 2102 includes an on-tool optical based ultrasound transducer 2104 configured to receive beacon signals and beamforming signals, as discussed herein. Received beacon signals and beamforming signals may be processed and used to assist in the navigation of the surgical tool 2102 to the appropriate location for kidney drainage.

In further embodiments, the systems, devices, and methods discussed herein may be employed for any type of rendezvous procedure. In any procedure where it is necessary for two different surgical tools to meet within the anatomy of the patient, the accurate tracking and guidance provided by the on-tool optical based ultrasound transducers described herein may provide improvements and advantages. One example of a rendezvous procedure includes a urology rendezvous procedure, wherein a needle and snare rendezvous within a patient kidney to allow a guidewire to be inserted. The guidewire may then be used to deliver another medical device, such as a urethral stent or other device.

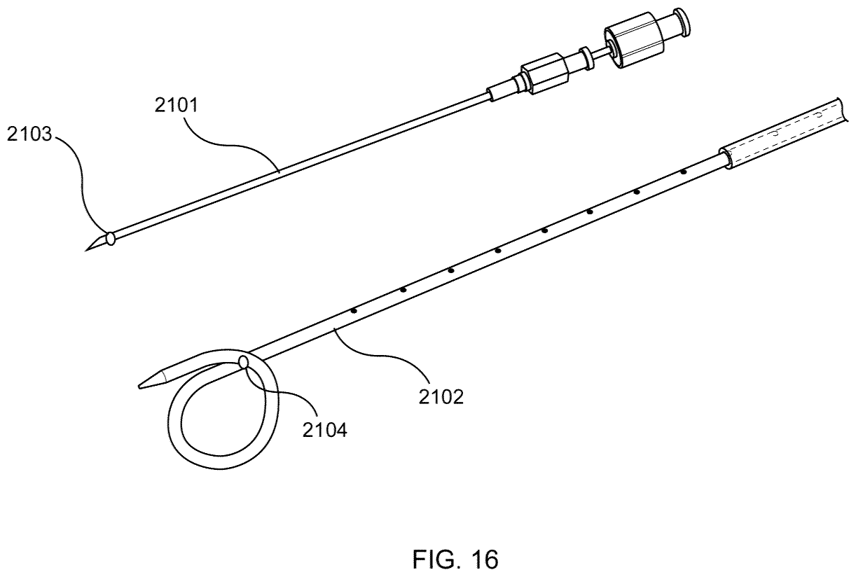

FIG. 17 illustrates a surgical tool 2201 that may be employed with such rendezvous procedures. The surgical tool 2201 is a snare and includes one or more on-tool optical based ultrasound transducers 2202. In embodiments, the on-tool optical based ultrasound transducers may be individual elements disposed on the snare loops 2203 and/or at a snare base 2204. In embodiments, polarization type optical based ultrasound transducers may be disposed along the length of the snare loops 2203 and may thus provide visualization and tracking of the snare itself. In embodiments, the fibers of polarization type optical based ultrasound transducers may themselves be configured to function as the snare loops 2203. Further tools that may be used with a urology rendezvous procedure include any of the needles and guidewires discussed herein.

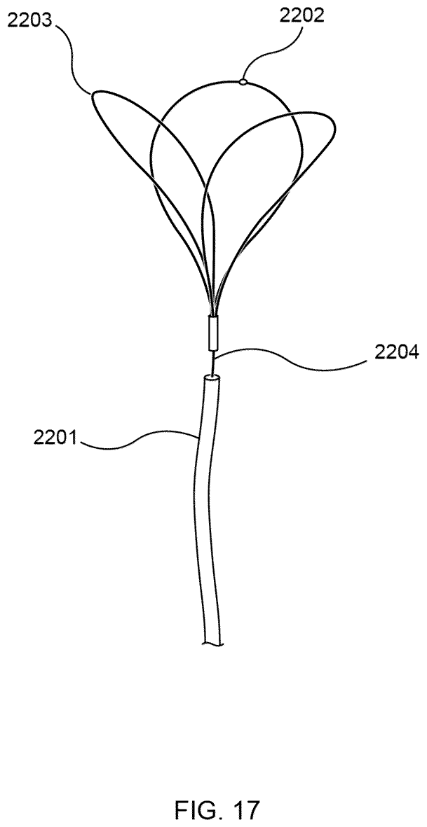

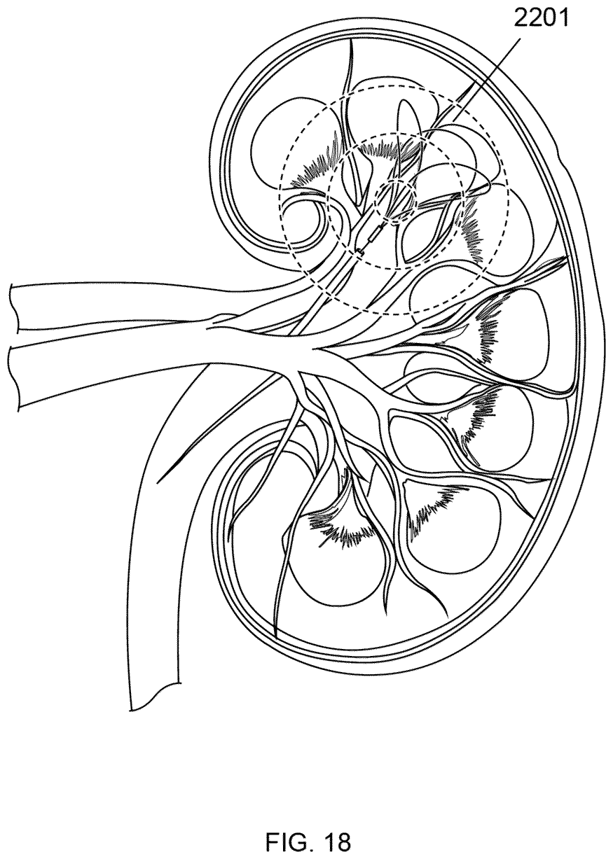

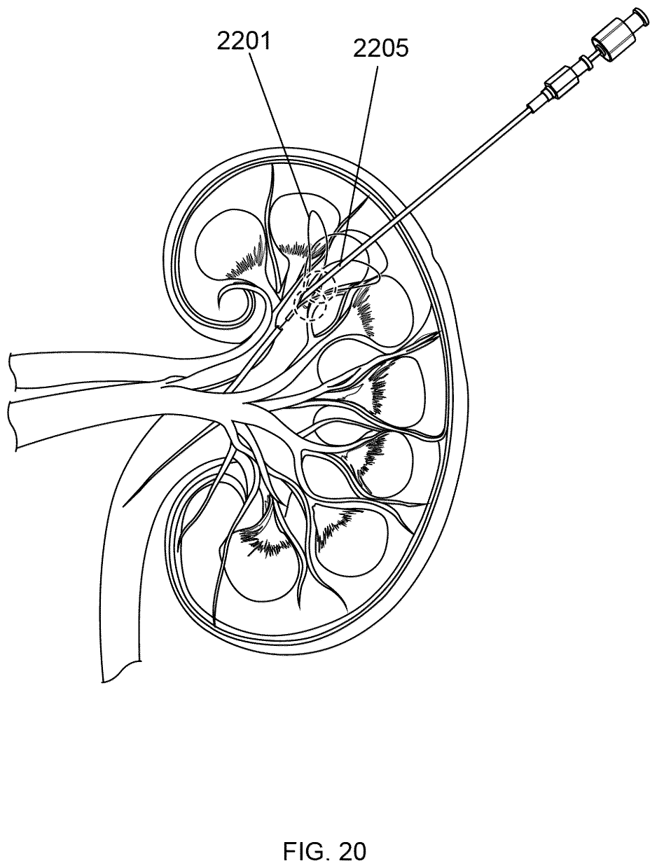

FIGS. 18-21 illustrate aspects of a urology rendezvous procedure. FIG. 18 illustrates a snare, such as surgical tool 2201 that has been guided to the interior of a patient kidney. FIG. 19 illustrates a needle 2205 under ultrasound being guided to the interior of the kidney to rendezvous with the snare 2201, using on-tool optical based ultrasound transducers on both the needle tool and the snare tool. FIG. 19 also displays location indicators 2206 and 2207 associated, respectively, with the needle 2205 and the snare 2201. FIG. 20 is a schematic of the kidney illustrating the rendezvous of the needle tool 2205 and the snare tool 2201. FIG. 21 illustrates guiding the snare tool 2201 to the needle tool 2205 with the respective location indicators 2206 and 2207. The snare tool may then be used to pull the guidewire further into the patient anatomy to the appropriate procedure location, for example, the urethra.

Use of on-tool optical based ultrasound transducer technology in a urology rendezvous procedure, or any type of rendezvous procedure, may provide superior accuracy to device tracking during such a procedure. Accordingly, these procedures may be performed in a reduced amount of time with greater accuracy and safety.

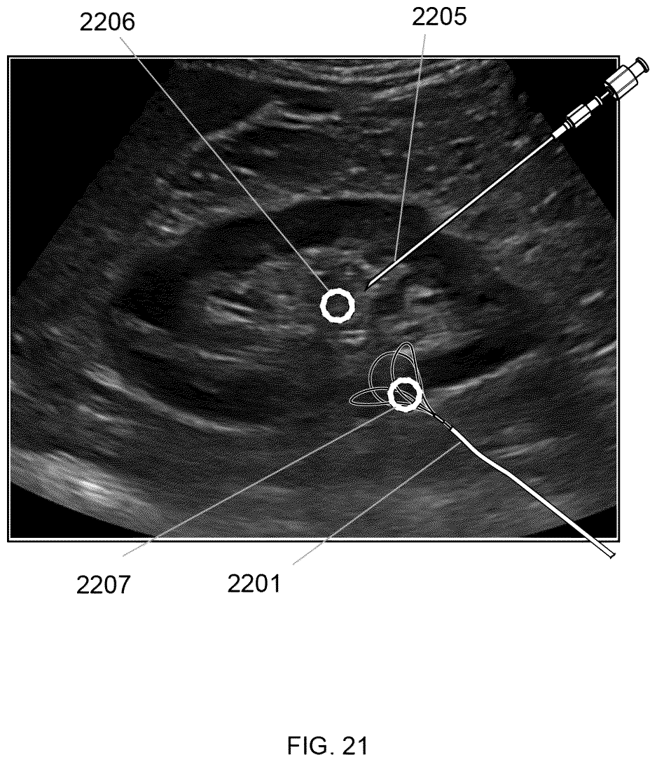

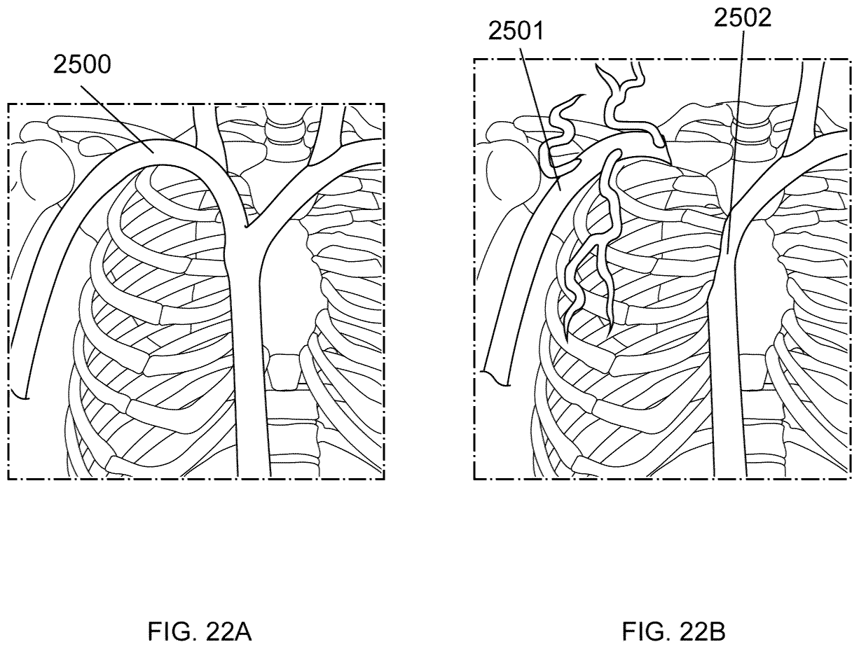

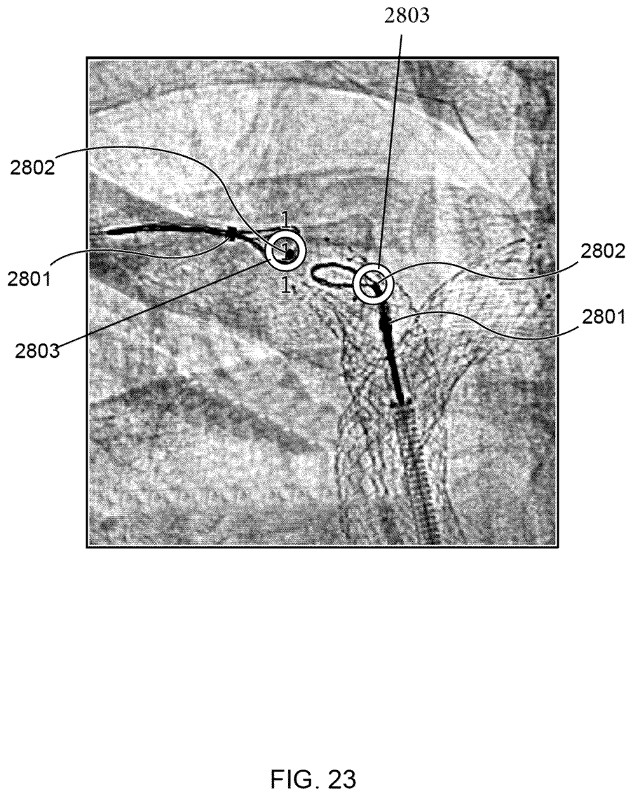

Another rendezvous procedure that may benefit from use of on-tool optical based ultrasound transducer technology described herein may include a stent recanalization procedure. FIG. 22A illustrates a patent 2500 vein and FIG. 22B illustrates an occluded vein. In FIG. 22B, portions 2501 and 2502 illustrate the vein on either side of the occlusion. A stent recanalization procedure may be implemented to clear the occlusion and implant a stent in the occlusion location to maintain the patency of the vein. Such procedures may involve techniques, such as dissection (blunt or not) techniques, that require surgical tools operating on either side of the occlusion location. Thes types of procedure are often performed under x-ray fluoroscopy and are often painstaking and time-consuming procedures. Frequently, there are many potential “danger zones” near the occlusion area and careful guidance and accurate tracking may have significant advantages. FIG. 23 illustrates the use of a pair of blunt dissection surgical tools 2801, each equipped with an on-tool optical based ultrasound transducer 2802. The respective location indicators 2803 are also illustrated. Surgical tool tracking methods and techniques described herein may be employed to guide each of the surgical tools 2801 to the occlusion site to perform the blunt dissection necessary to complete the recanalization procedure.

The previous discussion provides details of some procedures that may be improved through use of on-tool optical based ultrasound transducers. Additional procedures that may similarly benefit include any type of vascular access procedure, pain management procedures, different and varied RF, microwave, cryo ablation procedures, neurosurgical procedures, OB/GYN procedures, different and varied deep tissue biopsy procedures, prostate biopsy procedures, contrast enhanced ultrasound procedures, spinal fluid draw procedures, breast biopsy procedures, and others.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

Additional embodiments consistent with the present disclosure include the following.

Embodiment 1 is a system for medical device tracking, comprising: an ultrasound transducer configured to generate ultrasound beacon signals; a first instrument including a first optical ultrasound transducer configured to receive the ultrasound beacon signals and generate first optical signals corresponding to the ultrasound beacon signals; a second instrument including a second optical ultrasound transducer configured to receive the ultrasound beacon signals and generate second optical signals corresponding to the ultrasound beacon signals; at least one processor configured to: obtain first location signals corresponding to the first optical signals; obtain second location signals corresponding to the second optical signals; cause display of a first location indicator corresponding to a first location of the first instrument and a second location indicator corresponding to a second location of the second instrument.

Embodiment 2 is the system of embodiment 1, wherein the at least one processor is further configured to cause display of the first location indicator and the second location indicator on an anatomical map.

Embodiment 3 is the system of any of embodiments 1-2, wherein the ultrasound transducer is further configured to generate ultrasound beamforming signals to generate the anatomical map.

Embodiment 4 is the system of any of embodiments 1-3, wherein the at least one processor is further configured to cause display of a first in plane indicator corresponding to the first instrument.

Embodiment 5 is the system of any of embodiments 1-4, wherein the at least one processor is further configured to cause display of a rendezvous guide configured to guide the first instrument to a location of the second instrument.

Embodiment 6 is the system of any of embodiments 1-5, wherein the first location indicator and the second location indicator are provided relative to one another.

Embodiment 7 is the system of any of embodiments 1-6, wherein the first location indicator and the second location indicator are provided relative to an external reference framework.

Embodiment 8 is the system of any of embodiments 1-7, wherein the first optical ultrasound transducer is further configured to generate ultrasound images from the perspective of the first instrument.

Embodiment 9 is the system of any of embodiments 1-8, wherein: the first instrument includes a needle, the second instrument includes a guidewire, and the system is configured for performing a transjugular intrahepatic portosystemic shunt procedure.

Embodiment 10 is the system of any of embodiments 1-9, wherein: the first instrument includes a needle, the second instrument includes a catheter, and the system is configured for performing a nephrostomy procedure.

Embodiment 11 is the system of any of embodiments 1-10, wherein: the first instrument includes a needle, the second instrument includes a snare, and the system is configured for performing a nephrostomy procedure.

Embodiment 12 is the system of any of embodiments 1-11, wherein: the first instrument includes a first blunt dissection tool, the second instrument includes a second blunt dissection tool, and the system is configured for performing a recanalization procedure.

Embodiment 13 is a system for medical device guidance, comprising: an ultrasound transducer configured to generate ultrasound beacon signals; an instrument including an optical ultrasound transducer configured to receive the ultrasound beacon signals and generate optical signals corresponding to the ultrasound beacon signals; at least one processor configured to: obtain location signals corresponding to the optical signals; cause display of a location indicator corresponding to a location of the instrument.

Embodiment 14 is the system of embodiment 13, wherein the at least one processor is further configured to cause display of in plane indicator corresponding to the location of the instrument.

Embodiment 15 is the system of embodiments 13 or 14, wherein: the instrument further includes an ablation tip configured to provide ablation energy, and the optical ultrasound transducer is further configured to measure tissue temperature.

Embodiment 16 is the system of any of embodiments 13-15, wherein: the instrument further includes an ablation tip configured to provide ablation energy, and the at least one processor is configured to cause display of the location indicator during application of the ablation energy.

Embodiment 17 is a system for ablation tool guidance, comprising: an ultrasound transducer configured to generate ultrasound beacon signals; an instrument including an optical ultrasound transducer configured to receive the ultrasound beacon signals and generate optical signals corresponding to the ultrasound beacon signals; at least one processor configured to: obtain location signals corresponding to the optical signals; cause display of a location indicator corresponding to a location of the instrument.

1. A system for medical device tracking, comprising:
an ultrasound transducer configured to generate ultrasound beacon signals;
a first instrument including a first optical ultrasound transducer configured to receive the ultrasound beacon signals and generate first optical signals corresponding to the ultrasound beacon signals;
a second instrument including a second optical ultrasound transducer configured to receive the ultrasound beacon signals and generate second optical signals corresponding to the ultrasound beacon signals; and
at least one processor configured to:
obtain first location signals corresponding to the first optical signals,
obtain second location signals corresponding to the second optical signals, and
cause display of a first location indicator corresponding to a first location of the first instrument and a second location indicator corresponding to a second location of the second instrument.

2. The system of claim 1, wherein the at least one processor is further configured to cause display of the first location indicator and the second location indicator on an anatomical map.

3. The system of claim 2, wherein the ultrasound transducer is further configured to generate ultrasound beamforming signals to generate the anatomical map.

4. The system of claim 1, wherein the at least one processor is further configured to cause display of a first in plane indicator corresponding to the first instrument.

5. The system of claim 1, wherein the at least one processor is further configured to cause display of a rendezvous guide configured to guide the first instrument to a location of the second instrument.

6. The system of claim 1, wherein the at least one processor is further configured to cause display of an orientation indicator corresponding to the first instrument.

7. The system of claim 1, wherein the at least one processor is further configured to cause display of a path history indictor corresponding to the first instrument.

8. The system of claim 1, wherein the at least one processor is further configured to cause display of a trajectory indicator corresponding to the first instrument.

9. The system of claim 1, wherein the first location indicator and the second location indicator are provided relative to one another.

10. The system of claim 1, wherein the first location indicator and the second location indicator are provided relative to an external reference framework.

11. The system of claim 1, wherein the first optical ultrasound transducer is further configured to generate ultrasound images from a perspective of the first instrument.

12. The system of claim 1, wherein:
the first instrument includes a needle,
the second instrument includes a guidewire, and
the system is configured for performing a transjugular intrahepatic portosystemic shunt procedure.

13. The system of claim 1, wherein:
the first instrument includes a needle,
the second instrument includes a catheter, and
the system is configured for performing a nephrostomy procedure.

14. The system of claim 1, wherein:
the first instrument includes a needle,
the second instrument includes a snare, and
the system is configured for performing a nephrostomy procedure.

15. The system of claim 1, wherein:
the first instrument includes a first blunt dissection tool,
the second instrument includes a second blunt dissection tool, and
the system is configured for performing a recanalization procedure.

16. A system for medical device guidance, comprising:
an ultrasound transducer configured to generate ultrasound beacon signals;
an instrument including an optical ultrasound transducer configured to receive the ultrasound beacon signals and generate optical signals corresponding to the ultrasound beacon signals; and
at least one processor configured to:
obtain location signals corresponding to the optical signals;
cause display of a location indicator corresponding to a location of the instrument.

17. The system of claim 16, wherein the at least one processor is further configured to cause display of in plane indicator corresponding to the location of the instrument.

18. The system of claim 16, wherein:
the instrument further includes an ablation tip configured to provide ablation energy, and
the optical ultrasound transducer is further configured to measure tissue temperature.

19. The system of claim 16, wherein:
the instrument further includes an ablation tip configured to provide ablation energy, and
the at least one processor is configured to cause display of the location indicator during application of the ablation energy.

20. A system for ablation tool guidance, comprising:
an ultrasound transducer configured to generate ultrasound beacon signals;
an instrument including an optical ultrasound transducer configured to receive the ultrasound beacon signals and generate optical signals corresponding to the ultrasound beacon signals; and
at least one processor configured to:
obtain location signals corresponding to the optical signals;
cause display of a location indicator corresponding to a location of the instrument.
